(19) United States
(12) Patent Application Publication
(10) Pub. No.: US 2026/0035755 A1
(43) Pub. Date: Feb. 5, 2026

(54) METHOD FOR COLORECTAL CANCER USING FECAL MICROBIOME PROFILING

(71) Applicants: BARCELONA SUPERCOMPUTING CENTER – CENTRO NACIONAL DE SUPERCOMPUTACIÓN, Barcelona (ES); FUNDACIÓ INSTITUT DE RECERCA BIOMÉDICA (IRB BARCELONA), Barcelona (ES); INSTITUCIÓ CATALANA DE RECERCA I ESTUDIS AVANÇATS, Barcelona (ES); FUNDACIO DE RECERCA CLÍNIC BARCELONA – INSTITUT D’INVESTIGACIONS BIOMÈDIQUES AUGUST PI I SUNYER, Barcelona (ES)

(72) Inventors: Toni Gabaldón, Barcelona (ES); Olfat Khannous, Barcelona (ES); Ester Saus, Barcelona (ES); Sergi Castellví, Barcelona (ES)

(21) Appl. No.: 18/875,260

(22) PCT Filed: Jun. 16, 2023
(86) PCT No.: PCT/EP2023/066277

§ 371 Date: Dec. 16, 2024

(30) Foreign Application Priority Data
Jun. 17, 2022 (EP) 22179747.5

(51) Int. Cl. C12Q 1/689; G16B 25/10; G16B 40/20

(52) U.S. Cl. CPC C12Q 1/689; G16B 25/10; G16B 40/20; C12Q 2600/118

(57) ABSTRACT

The invention relates to a two-phase method for screening for colorectal cancer (CRC) using fecal microbiome profiling. The method comprises determining in a fecal sample isolated from the subjects the levels of two or more bacterial taxa. classifying with a computer algorithm in a first phase CRC samples vs. non-CRC samples and classifying with a computer algorithm in a second phase the samples that are classified as being non-CRC in the first phase into clinically relevant (CR) samples and non-CR samples using two or more bacterial taxa that are differentially abundant in CR samples relative to non-CR samples. The invention also relates to a kit comprising reagents for conducting the method and a computer program.

### CROSS-REFERENCE TO RELATED APPLICATIONS

This Application is a U.S. National Stage Application of PCT/EP2023/066277, filed Jun. 16, 2023, which claims priority to European Patent Publication No. 22179747.5, filed Jun. 17, 2022, both of which are incorporated by reference in their entireties.

### SEQUENCE LISTING

The Instant Application contains a Sequence Listing which has been submitted electronically in XML format and is hereby incorporated by reference in its entirety. Said XML copy, created on Oct. 15, 2025, is named “CLK0042US” and is 5,423 bytes in size. The Sequence Listing does not go beyond the disclosure in the application as filed.

### FIELD OF THE INVENTION

The present invention belongs to the field of medicine. More specifically it relates to a method for screening for colorectal cancer using fecal microbiome profiling.

### BACKGROUND OF THE INVENTION

Colorectal cancer (CRC) is the third most common cancer type and the second leading cause of cancer-related deaths worldwide (1), accounting for nearly 900,000 deaths each year. CRC presents different molecular phenotypes and a strong resistance to therapies. It has been suggested that this malignant disease develops from the pathological transformation of normal colonic epithelium to adenomatous polyps, which ultimately leads to invasive cancer. This process is gradual and involves the accumulation of genetic and/or epigenetic alterations (2). Non-environmental risk factors in CRC include age and genetic susceptibility (3).

The incidence of CRC increases with economic development and Westernization of dietary and lifestyle habits, which hints at a significant effect of environmental and lifestyle factors, which likely act in combination with genetic predisposition (4). In this regard a growing body of evidence has linked alterations of the gastrointestinal tract microbiota with CRC development (5).

Earlier research has shown that alterations in the gut microbiota may influence colon tumorigenesis (6) through chronic inflammation or the production of carcinogenic compounds (7). Differences in the relative abundances of some microbial species or genera have been found when comparing paired tumor and normal tissues, or fecal samples from CRC patients and healthy subjects (8,9). Diagnosis of CRC is challenging and involves a complex process that usually starts with the detection of the first symptoms by the patient, and is followed by clinical diagnostic procedures, mainly based on colonoscopy.

The implementation of preventive measures and early diagnosis of CRC can save many lives (10,11), and routine screening of populations above a certain age has been implemented in many countries. Current CRC screening consists of a two-step procedure with a non-invasive test (most commonly a fecal immunochemical test (FIT) quantification of occult hemoglobin in the stool) followed by colonoscopy if the test is positive (FIT-positive, at an assigned threshold hemoglobin concentration) (12,13). This approach is effective but results in a high rate of false positives at the first step and many unnecessary colonoscopies (only about 20-30% of colonoscopies performed in FIT-positive individuals reveal clinically relevant features, and only 3-5% CRC) (14).

Colonoscopy is an invasive, expensive and time-consuming procedure, and hence additional biomarkers that could better stratify individuals with higher risk for CRC and risk-associated premalignant lesions to undergo a colonic examination would significantly reduce health-care costs.

Much current research is directed towards finding additional criteria, such as risk factors and other biomarkers to be considered by the decision algorithms used to personalize positive FIT testing to colonoscopy. These include consideration of molecular biomarkers related to the processes underlying colorectal carcinogenesis from circulating tumor cells (15), cell-free DNA (16), microRNAs (17), as well as metabolites from plasma (18) samples, and germline risk genetic variants from blood DNA (19). Given the growing evidence for the existence of microbiome alterations associated with CRC, and the likely involvement of the microbiota in the origin and progression of cancer (5,21), microbial markers have recently emerged as a promising additional factor to be considered in early screenings.

In addition, a better knowledge of the role of the gut metabolism, microbiota and microbiota-host interactions in the initiating stages of CRC may help establish preventive measures such as changes in diet or the use of pro- or prebiotics.

All in all, there is a need for early diagnosis non-invasive techniques to diagnose this malignant disease and allow the greatest prognosis and quality of life for the patients.

### DESCRIPTION

### Brief Description of the Invention

The present invention discloses an innovative approach for the early detection of Colorectal Cancer (CRC) that combines the microbiome profiling of a sample with a two-phase Al-based classifying algorithm designed to reduce the number of unnecessary colonoscopies and the early detection of clinically relevant cases to provide better prognosis for CRC patients.

To search for potential predictive biomarkers present in FIT and other type of fecal samples and to shed light on the potential roles of the gut microbiome in CRC development, it was performed microbiome profiling using targeted sequencing of the 16S rRNA gene V3-V4 region from DNA extracted directly from FIT tubes collected within the population screening program implemented in Catalonia, Spain (22).

A total of 2,889 FIT-positive samples and 246 FIT-negative samples were analyzed; their microbial composition and metabolic potential was assessed, and it was studied how they varied across samples with different colonoscopy results.

Significant differences in particular taxa and metabolic pathways among relevant stages of CRC development along the path from healthy tissue to carcinoma were found. Using diagnostic evaluations from colonoscopy, it has been reconstructed changes in the composition, taxon co-occurrence and metabolic features of microbial communities associated to clinically relevant traits such as the presence of polyps or distinct precancerous lesions, hinting to potential microbial roles in the origin and progression of CRC.

Finally, a machine learning algorithm was used to develop and validate a two-phase classifier that combines information from bacterial signatures, sex, age and hemoglobin) with high sensitivity that would help limit unnecessary colonoscopies while minimizing false negative rates (FIG. 1). This classifier achieved close to 100% sensitivity for CRC, while significantly reducing the current false positive rate.

The present invention relates to a method as defined in the claims.

In a first embodiment, the disclosure refers to a method for diagnosing a subject to suffer from colorectal cancer (CRC) or classifying a subject to have higher risk for developing CRC in a patient cohort comprising:
(i) determining in a fecal sample isolated from a subject the levels of three or more bacterial taxa;
(ii) classifying with a computer algorithm in a first phase CRC samples vs. non-CRC samples using two or more bacterial taxa that are differentially abundant in CRC samples relative to non-CRC samples, the hemoglobin content of the sample, and the age and sex of the donor;
(iii) classifying with a computer algorithm in a second phase the samples that are classified as being non-CRC in the first phase into clinically relevant (CR) samples and non-CR samples using two or more bacterial taxa that are differentially abundant in CR samples relative to non-CR samples, the hemoglobin content of the sample, and the age and sex of the donor, wherein CR comprises intermediate risk lesions, high risk lesions, carcinoma in situ (CIS), and Colorectal cancer (CRC);
   wherein the three or more bacterial taxa in step (i) are selected from the group consisting of Hungatella spp. Colinsella spp., Tyzzerella spp., Phascolarctobacterium succinatutens, Lactobacillus spp., Akkermansia spp., Akkermansia muciniphila, O. Mollicutes_RF39.UCF, Ruminococcaceae_UCG.002 spp., Ruminococcaceae_UCG.0010 spp., Odoribacter spp., O. Rhodospirillales.UCF, Victivallis spp., Ruminococcaceae_UCG.005 spp., Negativibacillus spp., Christensenellaceae_R.7 group spp., Oxalobacter spp., Butyrivibrio spp., Family_XIII_UCG.001 spp., Gemella spp., Peptostreptococcus spp., Pediococcus spp., Lactobacillus vaginalis, Enorma massiliensis, Megamonas funiformis, Peptostreptococcus anaerobius, Peptoniphilus lacrimalis, Lactobacillus oris, Alloscardovia omnicolens, Allisonella histaminiformans, Acidaminococcus fermatans, Collinsella bouchesdurhonensis, Corynebacterium spp., Veillonella dispar, Ezakiella spp., O. Chloroplast.UCF, Sphingomonas spp., Dialister succinatiphilus, Finegoldia magna, Bacteroides coprophilus, Eggerthella spp., Acidaminococcus spp., Enterococcus spp., Sutterella wadsworthensis, Bacteroides fragilis, Bacteroides plebeius, Bacteroides coprocola, Bifidobacterium longum, Bilofila spp., Parabacteroides merdae, DTU08 spp., Oscillibacter spp., Parabacteroides goldsteinii, Parabacteroides spp., Bacteroides spp., Coprobacter secundus, Prevotella timonensis, Streptococcus parasanguinis, Peptostreptococcus anaerobius, Streptococcus sobrinus, Lachnospiraceae_FCS020 group bacterium, Bifidobacterium dentium, Porphyromonas spp., Lachnospiraceae_UCC.008 spp., Enterobacter spp., Hungatella hathewayi, Ezakiella spp., Leukonostoc spp., Parabacteroides johnsonii, Bacteroides finegoldii, Eisenbergiella spp., Alistipes finegoldii, F. Erysipelotrichaceae.UCG, Dorea formicigenerans, Bacteroides caccae, Fusobacterium.unclassified.S106, Peptostreptococcus.unclassified.S87, Erysipelotrichaceae_UCG.003.unclassified.S297, Alistipes.putredinis, Prevotella.unclassified.S33 and Coprococcus.comes.

In a second embodiment, the disclosure refers to a kit comprising:
(a) reagents for conducting a method for determining the presence or the abundance of the bacteria in a fecal sample to determine the levels of two or more bacterial taxa in step (i) of the method of the first embodiment; and
(b) a computer program stored on a computer-readable data carrier or chip, comprising instructions which, when the program is executed by a computer, cause the computer to carry out steps (ii) and (iii) of the method of the first embodiment.

### DESCRIPTION OF THE FIGURES

The following Figures are merely illustrative of the present invention and should not be construed to limit the scope of the invention as indicated by the appended claims in any way. The figures show:

FIG. 1: Summary of the general scheme of the screening method.

FIG. 2: Flow chart of the two-phase classification procedure. FIT positive samples are subjected to microbiome profiling by 16S rRNA sequencing. Then a two-phase classifier is applied: first the algorithm classifies CRC vs non-CRC samples. Samples that are classified as non-CRC in the first phase are subjected to a second model that classifies CR vs non-CR samples. FIT: Fecal immunochemical test; CRC: Colorectal cancer: CR: Clinically relevant.

FIG. 3: Pie Chart representing the 10 most abundant genera of studied CRIPREV samples (FIT positive samples). The other genera were grouped and named as “Others”.

FIG. 4: Comparison of FIT positive 16S samples, stool 16S and WGS samples from the same individuals. A) Correlation matrix showing only significant correlations. The darker the color, the more correlated the samples. B) Multidimensional plot (MDS) representing the Aitchison distance, revealed a grouping of the samples according to the source and sequencing of the samples. Samples were colored according to the source and sequencing methodology and shaped according to the id of the sample, to match samples from the same individuals.

FIG. 5: Alpha diversity characterization of the FIT positive samples. The lines inside the boxplots represent the medians for each of the groups. Statistical test: Kruskall-Wallis or Wilcoxon test, with a significant result when p<0.05. A) Observed index according to the diagnosis (Carcinoma in situ (CIS), Colorectal cancer (CRC), lesion that is not associated to risk (LNAR), high risk lesion (HRL), low risk lesion (LRL), intermediate risk lesion (IRL) or Negative (N) samples) and Risk (clinically relevant (CR) vs non-clinically relevant (non-CR) samples) variables. B) Shannon and Simpson indices according to the diagnosis.

FIG. 6: MDS plots using Aitchison distance. The samples are identified as *, Δ, □, +, or [symbol] , according to the diagnosis. 95% confidence ellipses are represented for each of the diagnosed groups.

FIG. 7: Differential abundance analysis from FIT positive samples. Representation of the 34 bacterial species found as significantly differentially abundant between groups of diagnoses following the path from healthy colon to colorectal cancer. Different colonoscopy diagnoses are depicted from left to right following this path with healthier states at the left and in the following order: N, Negative; LNAR, Lesion not associated to risk; LRL, Low risk lesion; IRL, Intermediate risk lesion; HRL, High risk lesion; CIS, Carcinoma in situ; CRC, Colorectal cancer. Lines connecting different diagnoses indicate comparisons, with differentially abundant species names indicated.

FIG. 8: Effect size of species found as significantly differentially abundant when comparing CRC vs non-CRC samples (A) and CR vs non-CR samples (B). Bars are grey for overrepresentation and black for underrepresentation. The bars are sorted according to the effect size. In bold are highlighted the taxa that appeared as differentially abundant in both comparisons.

FIG. 9: Potential selection (Number of models selected/Number of evaluated models, in % of the different feature selection methods.

FIG. 10: For each of the studied taxa: Number of models in which the taxa was included, and number of models selected (for the numbers see TABLE 8).

FIG. 11: Percentage of saved colonoscopies and clinically relevant sensitivity according to the different specifications of the proposed classifier.

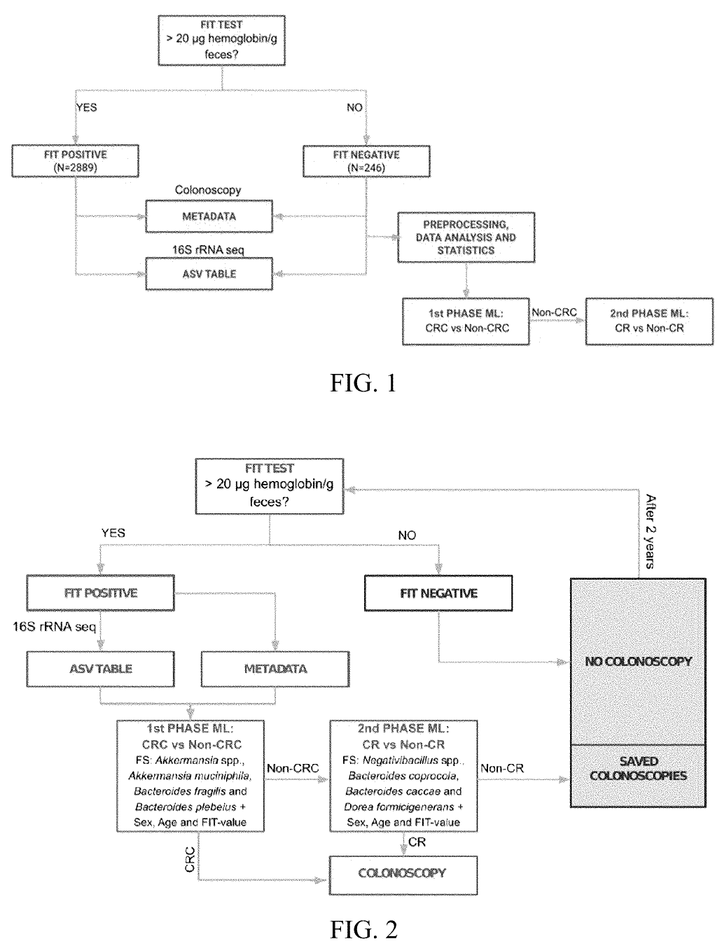

All_taxa: All the intersecting taxa between the CRIPREV and the validation datasets were used as features.

DA_taxa: All the intersecting differentially abundant taxa between the CRIPREV and the validation datasets were used as features.

4-4 taxa panel: 4 taxa panel for each of the phases.

4-4 taxa panel, adjW: 4 taxa panel for each of the phases, with less penalization of the CR samples in the second phase.

FIT_filter_4-4 taxa panel: Samples above 954 of the FIT value (μg hemoglobin/g feces) were directed to colonoscopy and the remaining samples were subjected to the classifier.

FIT_filter_4-4 taxa panel_adjW: Samples above 954 of the FIT value (μg hemoglobin/g feces) were directed to colonoscopy and the remaining samples were subjected to the classifier. Less penalization of the CR samples in the second phase.

### DETAILED DESCRIPTION OF THE INVENTION

### Definitions

In the following the invention is described in more detail with reference to the figures. The described specific embodiments of the invention, examples, or results are, however, intended for illustration only and should not be construed to limit the scope of the invention as indicated by the appended claims in any way.

It is to be understood that this invention is not limited to the particular methodology, protocols, and reagents described herein as these may vary. It is also to be understood that the terminology used herein is to describe particular embodiments only and is not intended to limit the scope of the present invention which will be limited only by the appended claims. Unless defined otherwise, all technical and scientific terms used herein have the same meanings as commonly understood by one of ordinary skill in the art.

Each of the documents cited in this specification (including all patents, patent applications, scientific publications, manufacturer's specifications, instructions, etc.), whether supra or infra, is hereby incorporated by reference in its entirety. In the event of a conflict between the definitions or teachings of such incorporated references and definitions or teachings recited in the present specification, the text of the present specification takes precedence.

The term “comprising” or variations thereof such as “comprise(s)” according to the present invention (especially in the context of the claims) is to be construed as an open-ended term or non-exclusive inclusion, respectively (i.e., meaning “including, but not limited to,”) unless otherwise noted. The term “comprising” shall encompass and include the more restrictive terms “consisting essentially of” or “comprising substantially”, and “consisting of”.

In the case of chemical compounds or compositions, the terms “consisting essentially of” or “comprising substantially” mean that specific further components can be present, namely those not materially affecting the essential characteristics of the compound or composition, e.g., unavoidable impurities.

The terms “a”, “an”, and “the” as used herein in the context of describing the invention (especially in the context of the claims) should be read and understood to include at least one element or component, respectively, and are to be construed to cover both the singular and the plural, unless otherwise indicated herein or clearly contradicted by context.

In addition, unless expressly stated to the contrary, the term “or” refers to an inclusive “or” and not to an exclusive “or” (i.e., meaning “and/or”).

The phrase “selected from the group consisting of” means that one or more member(s) of the group is/are used and in any combination(s).

All numeric values are herein assumed to be modified by the term “about”, whether or not explicitly indicated. Recitation of ranges of values herein is merely intended to serve as a shorthand method of referring individually to each separate value falling within the range, unless otherwise indicated herein, and each separate value is incorporated into the specification as if it were individually recited herein.

The use of terms “for example”, “e.g.,”, “such as”, or variations thereof is intended merely to better illuminate the invention and does not pose a limitation on the scope of the invention unless otherwise claimed. These terms should be interpreted to mean “but not limited to” or “without limitation”.

The term “spp.” means an unclassified bacteria species from the same bacteria genus, e.g., Akkermansis spp. means an unclassified Akkermansia species. Alternatively, the term “unclassified” is used herein to indicate an unclassified bacteria species from the same bacteria genus. Thus, “spp.” and “unclassified” have the same meaning, and both terms are used herein equally.

The term “level(s) of bacteria” means relative abundance of a given bacterial taxa with respect to others present in the same sample.

The term “bacterial profile” means a set of relative abundances of bacterial taxa for a given sample.

The term “taxa” means a member of a taxonomic rank and comprises, e.g., a family, a genus, or a species of bacteria.

The term “FIT value” means the hemoglobin content, i.e., μg hemoglobin/g feces. The term “fecal immunochemical test” or “FIT” means any fecal test to determine occult hemoglobin in the stool by immunochemistry, for instance, a fecal immunochemistry tub (FIT) or fecal occult blood (iFOB).

The term “clinical relevant” (CR) means a defined grouping of risk stages in the development of CRC, including intermediate risk lesions (IRL), high risk lesions (HRL), carcinoma in situ (CIS), and colorectal cancer (CRC), but not negative/healthy (N), lesions not associated to risk (LNAR) and low risk lesions (LRL).

The term “CRIPREV” means a research project on the Catalan CRC Screening Program from which the samples for this invention were received. CriPrev: Prevention of colorectal cancer in the average-risk population using genomics biomarkers and microbiomics. Funded by PERIS, Generalitat de Catalunya (reference: SLT002/16/00398).

The term “method for determining the presence or abundance of bacteria” means by any method or protocol that is used for determining the presence or abundance of bacteria including sequencing of PCR from gene amplicons such as the 16S rRNA gene, Whole shotgun sequencing, cell-based methods such as the flow cytometry, quantitative PCR (qPCR), proteomics and antibody-based detection methods.

No language in this specification should be construed as indicating any non-claimed element as essential to the practice of the invention.

### Embodiments

In a first aspect, the present disclosure relates to a method for diagnosing a subject to suffer from colorectal cancer (CRC) or classifying a subject to have higher risk for developing CRC in a patient cohort, the method comprising:
(i) determining in a fecal sample isolated from a subject in a patient cohort the level of two or more bacterial taxa;
(ii) classifying with a computer algorithm in a first phase, CRC samples vs. non-CRC samples using two or more bacterial taxa that are differentially abundant in CRC samples relative to non-CRC samples, the hemoglobin content of the sample, the age and the sex of the donor;
(iii) classifying with a computer algorithm in a second phase, the samples that were classified as non-CRC in the first phase into clinically relevant (CR) samples and non-CR samples, using two or more bacterial taxa that are differentially abundant in CR samples relative to non-CR samples, the hemoglobin content of the sample, the age and the sex of the donor, wherein CR comprises intermediate risk lesions, high risk lesions, carcinoma in situ (CIS), and CRC;
wherein the two or more bacterial taxa are selected from the group consisting of Hungatella spp. Colinsella spp., Tyzzerella spp., Phascolarctobacterium succinatutens, Lactobacillus spp., Akkermansia spp., Akkermansia muciniphila, O. Mollicutes_RF39.UCF, Ruminococcaceae_UCG.002 spp., Ruminococcaceae_UCG.0010 spp., Odoribacter spp., O. Rhodospirillales.UCF, Victivallis spp, Ruminococcaceae_UCG.005 spp., Negativibacillus spp., Christensenellaceae_R.7_group spp., Oxalobacter spp., Butyrivibrio spp., Family_XIII_UCG.001 spp., Gemella spp., Peptostreptococcus spp., Pediococcus spp., Lactobacillus vaginalis, Enorma massiliensis, Megamonas funiformis, Peptostreptococcus anaerobius, Peptoniphilus lacrimalis, Lactobacillus oris, Alloscardovia omnicolens, Allisonella histaminiformans, Acidaminococcus fermatans, Collinsella bouchesdurhonensis, Corynebacterium spp., Veillonella dispar, Ezakiella spp., O. Chloroplast.UCF, Sphingomonas spp., Dialister succinatiphilus, Finegoldia magna, Bacteroides coprophilus, Eggerthella spp., Acidaminococcus spp., Enterococcus spp., Sutterella wadsworthensis, Bacteroides fragilis, Bacteroides plebeius, Bacteroides coprocola, Bifidobacterium longum, Bilofila spp., Parabacteroides merdae, DTU08 spp., Oscillibacter spp., Parabacteroides goldsteinii, Parabacteroides spp., Bacteroides spp., Coprobacter secundus, Prevotella timonensis, Streptococcus parasanguinis, Peptostreptococcus anaerobius, Streptococcus sobrinus, Lachnospiraceae_FCS020 group bacterium, Bifidobacterium dentium, Porphyromonas spp., Lachnospiraceae_UCC.008 spp., Enterobacter spp., Hungatella hathewayi, Ezakiella spp., Leukonostoc spp., Parabacteroides johnsonii, Bacteroides finegoldii, Eisenbergiella spp., Alistipes finegoldii, F. Erysipelotrichaceae.UCG, Dorea formicigenerans, and Bacteroides caccae.

In another aspect, the present disclosure relates to a method for diagnosing a subject to suffer from colorectal cancer (CRC) or classifying a subject to have higher risk for developing CRC in a patient cohort, the method comprising:
(i) determining in a fecal sample isolated from a subject in a patient cohort the level of three or more bacterial taxa;
(ii) classifying with a computer algorithm in a first phase, CRC samples vs. non-CRC samples using two or more bacterial taxa that are differentially abundant in CRC samples relative to non-CRC samples, the hemoglobin content of the sample, the age and the sex of the donor;
(iii) classifying with a computer algorithm in a second phase, the samples that were classified as non-CRC in the first phase into clinically relevant (CR) samples and non-CR samples, using two or more bacterial taxa that are differentially abundant in CR samples relative to non-CR samples, the hemoglobin content of the sample, the age and the sex of the donor, wherein CR comprises intermediate risk lesions, high risk lesions, carcinoma in situ (CIS), and CRC;
wherein the two or more bacterial taxa are selected from the group comprising Hungatella spp. Colinsella spp., Tyzzerella spp., Phascolarctobacterium succinatutens, Lactobacillus spp., Akkermansia spp., Akkermansia muciniphila, O. Mollicutes_RF39.UCF; Ruminococcaceae_UCG.002 spp., Ruminococcaceae_UCG.0010 spp., Odoribacter spp., O. Rhodospirillales.UCF, Victivallis spp, Ruminococcaceae_UCG.005 spp., Negativibacillus spp., Christensenellaceae_R.7_group spp., Oxalobacter spp., Butyrivibrio spp., Family_XIII_UCG.001 spp., Gemella spp., Peptostreptococcus spp., Pediococcus spp., Lactobacillus vaginalis, Enorma massiliensis, Megamonas funiformis, Peptostreptococcus anaerobius, Peptoniphilus lacrimalis, Lactobacillus oris, Alloscardovia omnicolens, Allisonella histaminiformans, Acidaminococcus fermatans, Collinsella bouchesdurhonensis, Corynebacterium spp., Veillonella dispar, Ezakiella spp., O. Chloroplast.UCF, Sphingomonas spp., Dialister succinatiphilus, Finegoldia magna, Bacteroides coprophilus, Eggerthella spp., Acidaminococcus spp., Enterococcus spp., Sutterella wadsworthensis, Bacteroides fragilis, Bacteroides plebeius, Bacteroides coprocola, Bifidobacterium longum, Bilofila spp., Parabacteroides merdae, DTU08 spp., Oscillibacter spp., Parabacteroides goldsteinii, Parabacteroides spp., Bacteroides spp., Coprobacter secundus, Prevotella timonensis, Streptococcus parasanguinis, Peptostreptococcus anaerobius, Streptococcus sobrinus, Lachnospiraceae_FCS020 group bacterium, Bifidobacterium dentium, Porphyromonas spp., Lachnospiraceae UCC.008 spp., Enterobacter spp., Hungatella hathewayi, Ezakiella spp., Leukonostoc spp., Parabacteroides johnsonii, Bacteroides finegoldii, Eisenbergiella spp., Alistipes finegoldii, F. Erysipelotrichaceae.UCG, Dorea formicigenerans, Bacteroides caccae, Fusobacterium.unclassified.S106, Peptostreptococcus.unclassified.S87, Erysipelotrichaceae_UCG.003.unclassified.S297, Alistipes.putredinis, Prevotella.unclassified.S33 and Coprococcus.comes.

In a more preferred embodiment, the taxa in any of steps ii) or iii) is selected from any of the following: Bacteroides.coprocola, Bifidobacterium.longum, Porphyromonas.unclassified.S30, Eisenbergiella.unclassified.S226, Peptostreptococcus.unclassified.S87, Negativibacillus.unclassified.S269, unclassified.unclassified.S306, Acidaminococcus.unclassified.S307, Bacteroides.coprocola, Bifidobacterium.longum, Odoribacter.unclassified.S27, Porphyromonas.unclassified.S30, Christensenellaceae_R.7_group.unclassified.S209, Eisenbergiella.unclassified.S226, Peptostreptococcus.unclassified.S87, Ruminococcaceae_UCG.005.unclassified.S92, and Akkermansia.unclassified.S361.

The skilled person appreciates that the phrase “classifying a patient with risks of development of colorectal cancer” includes the diagnosis of non-CRC and the diagnosis of different stages of CRC development, for example, negative (N), lesion not associated to risk (LNAR), low risk lesion (LRL), intermediate risk lesion (IRL), high risk lesion (HRL) and carcinoma in situ (CIS), and colorectal cancer (CRC). CRC is considered in both phases of the method because in order to achieve maximum sensitivity (error 0), misclassified CRCs may be included also in the second phase. This provides a second chance for the samples to be classified as clinically relevant in the model.

In a preferred embodiment, the fecal sample is a fecal immunochemical test (FIT) sample. The fecal sample of a patient is advantageously a sample used for a fecal immunochemical test (FIT). In a preferred embodiment, the fecal sample is a FIT-positive sample (i.e., having a hemoglobin content of >20 μg hemoglobin/g feces), because no additional fecal sample needs to be taken from a patient and stored for analysis. The method of the present invention allows significantly reducing the current false positive rate of the FIT. Of course, any stool sample can be used in the inventive method, and the inventive method is not limited to a FIT sample. In another preferred embodiment, the fecal sample is a FIT-negative sample (i.e., having a hemoglobin content of ≤20 μg hemoglobin/g feces).

According to the invention, the method comprises that in steps (ii) and (iii) the levels of two or more bacterial taxa are determined, preferably, three of more bacterial taxa. This is not to be understood as a limiting feature, i.e., in the invention levels of 4, 5, 6, 7 or even more combinations of taxa may be determined in each step if this is suitable or desired. It is understood that one of the two or more bacterial taxa in each step may coincide.

Examples for bacteria combinations whose levels are determined are bacteria combinations selected from the group consisting of (the meaning of the terms “taxadown”, taxatop”, “taxarandom” is explained in section “Combinations of taxa” further down).

In a more preferred embodiment, when a sample is FIT positive, the taxa is selected from any of the following combinations:

| | |
|---|---|
| 1 | Akkermansia.unclassified.S361, Akkermansia.muciniphila, |
| | Bacteroides.coprocola, Dorea.formicigenerans; |
| 2 | Akkermansia.unclassified.S361, Akkermansia.muciniphila, |
| | Bifidobacterium.longum, Dorea.formicigenerans; |
| 3 | Akkermansia.unclassified.S361, Akkermansia.muciniphila, |
| | Bifidobacterium.longum, unclassified.unclassified.S306; |
| 4 | Akkermansia.unclassified.S361, Akkermansia.muciniphila, |
| | Dorea.formicigenerans, unclassified.unclassified.S306; |
| 5 | Akkermansia.unclassified.S361, Akkermansia.muciniphila, |
| | Negativibacillus.unclassified.S269, Dorea.formicigenerans; |
| 6 | Akkermansia.unclassified.S361, Akkermansia.muciniphila, |
| | Negativibacillus.unclassified.S269, Alistipes.finegoldii; |
| 7 | Akkermansia.muciniphila, Bacteroides.plebeius, |
| | Negativibacillus.unclassified.S269, Bacteroides.coprocola; |
| 8 | Akkermansia.muciniphila, Bacteroides.plebeius, |
| | Bacteroides.coprocola, Bacteroides.caccae; |
| 9 | Akkermansia.muciniphila, Bacteroides.plebeius, |
| | Bifidobacterium.longum, Dorea.formicigenerans; |
| 10 | Akkermansia.muciniphila, Bacteroides.plebeius, |
| | Dorea.formicigenerans, unclassified.unclassified.S306; |
| 11 | Akkermansia.muciniphila, Bacteroides.plebeius, |
| | Negativibacillus.unclassified.S269, Dorea.formicigenerans; |
| 12 | Akkermansia.muciniphila, Bacteroides.fragilis, |
| | Bacteroides.coprocola, Bacteroides.caccae; |
| 13 | Akkermansia.muciniphila, Bacteroides.fragilis, |
| | Bifidobacterium.longum, Bacteroides.caccae; |
| 14 | Akkermansia.muciniphila, Bacteroides.fragilis, |
| | Bifidobacterium.longum, Dorea.formicigenerans; |
| 15 | Akkermansia.muciniphila, Bacteroides.fragilis, |
| | Bifidobacterium.longum, Alistipes.finegoldii; |
| 16 | Akkermansia.muciniphila, Bacteroides.fragilis, |
| | Bilophila.unclassified.S322, Bacteroides.caccae; |
| 17 | Akkermansia.muciniphila, Bacteroides.fragilis, |
| | Bilophila.unclassified.S322, Alistipes.finegoldii; |
| 18 | Akkermansia.muciniphila, Bacteroides.fragilis, |
| | Bacteroides.caccae, unclassified.unclassified.S306; |
| 19 | Akkermansia.muciniphila, Bacteroides.fragilis, |
| | Bacteroides.caccae, Alistipes.finegoldii; |
| 20 | Akkermansia.muciniphila, Bacteroides.fragilis, |
| | Dorea.formicigenerans, Alistipes.finegoldii; |
| 21 | Akkermansia.muciniphila, Sutterella.wadsworthensis, |
| | Bacteroides.coprocola, Alistipes.finegoldii; |
| 22 | Akkermansia.muciniphila, Sutterella.wadsworthensis, |
| | Bilophila.unclassified.S322, Dorea.formicigenerans; |
| 23 | Akkermansia.muciniphila, Sutterella.wadsworthensis, |
| | Bilophila.unclassified.S322, unclassified.unclassified.S306; |
| 24 | Akkermansia.muciniphila, Sutterella.wadsworthensis, |
| | Dorea.formicigenerans, Alistipes.finegoldii; |
| 25 | Akkermansia.muciniphila, Sutterella.wadsworthensis, |
| | Negativibacillus.unclassified.S269, Dorea.formicigenerans; |
| 26 | Akkermansia.unclassified.S361, unclassified.unclassified.S358, |
| | Bifidobacterium.longum, unclassified.unclassified.S306; |
| 27 | Akkermansia.unclassified.S361, unclassified.unclassified.S358, |
| | Bilophila.unclassified.S322, unclassified.unclassified.S306; |
| 28 | Ruminococcaceae_UCG.002.unclassified.S91, Bacteroides.fragilis, |
| | Bifidobacterium.longum, Alistipes.finegoldii; |
| 29 | Akkermansia.unclassified.S361, Ruminococcaceae_UCG.002.unclassified.S91, |
| | Bifidobacterium.longum, Bacteroides.caccae; |
| 30 | Akkermansia.unclassified.S361, Ruminococcaceae_UCG.002.unclassified.S91, |
| | Bifidobacterium.longum, unclassified.unclassified.S306; |
| 31 | Akkermansia.unclassified.S361, Ruminococcaceae_UCG.002.unclassified.S91, |
| | Bilophila.unclassified.S322, Dorea.formicigenerans; |
| 32 | Akkermansia.unclassified.S361, Ruminococcaceae_UCG.002.unclassified.S91, |
| | Bilophila.unclassified.S322, unclassified.unclassified.S306; |
| 33 | Akkermansia.unclassified.S361, Ruminococcaceae_UCG.002.unclassified.S91, |
| | Bacteroides.caccae, unclassified.unclassified.S306; |
| 34 | Akkermansia.unclassified.S361, Bacteroides.plebeius, |
| | Bacteroides.coprocola, Dorea.formicigenerans; |
| 35 | Akkermansia.unclassified.S361, Bacteroides.plebeius, |
| | Bifidobacterium.longum, Bacteroides.caccae; |
| 36 | Akkermansia.unclassified.S361, Bacteroides.plebeius, |
| | Bilophila.unclassified.S322, Dorea.formicigenerans; |
| 37 | Akkermansia.unclassified.S361, Bacteroides.plebeius, |
| | Bilophila.unclassified.S322, unclassified.unclassified.S306; |
| 38 | Akkermansia.unclassified.S361, Bacteroides.plebeius, |
| | Bilophila.unclassified.S322, Alistipes.finegoldii; |
| 39 | Akkermansia.unclassified.S361, Bacteroides.plebeius, |
| | Bacteroides.caccae, Dorea.formicigenerans; |
| 40 | Akkermansia.unclassified.S361, Bacteroides.plebeius, |
| | Dorea.formicigenerans, unclassified.unclassified.S306; |
| 41 | Akkermansia.unclassified.S361, Bacteroides.plebeius, |
| | Negativibacillus.unclassified.S269, Bacteroides.caccae; |
| 42 | Akkermansia.unclassified.S361, Bacteroides.plebeius, |
| | Negativibacillus.unclassified.S269, Dorea.formicigenerans; |
| 43 | Akkermansia.unclassified.S361, Bacteroides.fragilis, |
| | Bacteroides.coprocola, Bacteroides.caccae; |
| 44 | Akkermansia.unclassified.S361, Bacteroides.fragilis, |
| | Bacteroides.coprocola, Dorea.formicigenerans; |
| 45 | Akkermansia.unclassified.S361, Bacteroides.fragilis, |
| | Bacteroides.coprocola, Alistipes.finegoldii; |
| 46 | Akkermansia.unclassified.S361, Bacteroides.fragilis, |
| | Bifidobacterium.longum, Bilophila.unclassified.S322; |
| 47 | Akkermansia.unclassified.S361, Bacteroides.fragilis, |
| | Bifidobacterium.longum, Bacteroides.caccae; |
| 48 | Akkermansia.unclassified.S361, Bacteroides.fragilis, |
| | Bifidobacterium.longum, Dorea.formicigenerans; |
| 49 | Akkermansia.unclassified.S361, Bacteroides.fragilis, |
| | Bifidobacterium.longum, unclassified.unclassified.S306; |
| 50 | Akkermansia.unclassified.S361, Bacteroides.fragilis, |
| | Bifidobacterium.longum, Alistipes.finegoldii; |
| 51 | Akkermansia.unclassified.S361, Bacteroides.fragilis, |
| | Negativibacillus.unclassified.S269, Bifidobacterium.longum; |
| 52 | Akkermansia.unclassified.S361, Bacteroides.fragilis, |
| | Bilophila.unclassified.S322, unclassified.unclassified.S306; |
| 53 | Akkermansia.unclassified.S361, Bacteroides.fragilis, |
| | Bilophila.unclassified.S322, Alistipes.finegoldii; |
| 54 | Akkermansia.unclassified.S361, Bacteroides.fragilis, |
| | Bacteroides.caccae, Dorea.formicigenerans; |
| 55 | Akkermansia.unclassified.S361, Bacteroides.fragilis, |
| | Bacteroides.caccae, unclassified.unclassified.S306; |
| 56 | Akkermansia.unclassified.S361, Bacteroides.fragilis, |
| | Dorea.formicigenerans, unclassified.unclassified.S306; |
| 57 | Akkermansia.unclassified.S361, Bacteroides.fragilis, |
| | Dorea.formicigenerans, Alistipes.finegoldii; |
| 58 | Akkermansia.unclassified.S361, Bacteroides.fragilis, |
| | Negativibacillus.unclassified.S269, Bacteroides.caccae; |
| 59 | Akkermansia.unclassified.S361, Bacteroides.fragilis, |
| | Negativibacillus.unclassified.S269, Dorea.formicigenerans; |
| 60 | Akkermansia.unclassified.S361, Bacteroides.fragilis, |
| | Negativibacillus.unclassified.S269, unclassified.unclassified.S306; |
| 61 | Akkermansia.unclassified.S361, Sutterella.wadsworthensis, |
| | Negativibacillus.unclassified.S269, Bacteroides.coprocola; |
| 62 | Akkermansia.unclassified.S361, Sutterella.wadsworthensis, |
| | Bacteroides.coprocola, Dorea.formicigenerans; |
| 63 | Akkermansia.unclassified.S361, Sutterella.wadsworthensis, |
| | Bacteroides.coprocola, unclassified.unclassified.S306; |
| 64 | Akkermansia.unclassified.S361, Sutterella.wadsworthensis, |
| | Bacteroides.coprocola, Alistipes.finegoldii; |
| 65 | Akkermansia.unclassified.S361, Sutterella.wadsworthensis, |
| | Bifidobacterium.longum, Bacteroides.caccae; |
| 66 | Akkermansia.unclassified.S361, Sutterella.wadsworthensis, |
| | Bifidobacterium.longum, unclassified.unclassified.S306; |
| 67 | Akkermansia.unclassified.S361, Sutterella.wadsworthensis, |
| | Bifidobacterium.longum, Alistipes.finegoldii; |
| 68 | Akkermansia.unclassified.S361, Sutterella.wadsworthensis, |
| | Bilophila.unclassified.S322, Bacteroides.caccae; |
| 69 | Akkermansia.unclassified.S361, Sutterella.wadsworthensis, |
| | Bilophila.unclassified.S322, Dorea.formicigenerans; |
| 70 | Akkermansia.unclassified.S361, Sutterella.wadsworthensis, |
| | Bilophila.unclassified.S322, unclassified.unclassified.S306; |
| 71 | Akkermansia.unclassified.S361, Sutterella.wadsworthensis, |
| | Bilophila.unclassified.S322, Alistipes.finegoldii; |
| 72 | Akkermansia.unclassified.S361, Sutterella.wadsworthensis, |
| | Bacteroides.caccae, Dorea.formicigenerans; |
| 73 | Akkermansia.unclassified.S361, Sutterella.wadsworthensis, |
| | Dorea.formicigenerans, unclassified.unclassified.S306; |
| 74 | Akkermansia.unclassified.S361, Sutterella.wadsworthensis, |
| | Dorea.formicigenerans, Alistipes.finegoldii; |
| 75 | Akkermansia.unclassified.S361, Sutterella.wadsworthensis, |
| | unclassified.unclassified.S306, Alistipes.finegoldii; |
| 76 | Akkermansia.unclassified.S361, Sutterella.wadsworthensis, |
| | Negativibacillus.unclassified.S269, Bilophila.unclassified.S322; |
| 77 | Akkermansia.unclassified.S361, Sutterella.wadsworthensis, |
| | Negativibacillus.unclassified.S269, Bacteroides.caccae; |
| 78 | Akkermansia.unclassified.S361, Sutterella.wadsworthensis, |
| | Negativibacillus.unclassified.S269, Alistipes.finegoldii; |
| 79 | Akkermansia.muciniphila, unclassified.unclassified.S358, |
| | Negativibacillus.unclassified.S269, Bacteroides.coprocola; |
| 80 | Akkermansia.muciniphila, unclassified.unclassified.S358, |
| | Bacteroides.coprocola, Dorea.formicigenerans; |
| 81 | Akkermansia.muciniphila, unclassified.unclassified.S358, |
| | Bifidobacterium.longum, Dorea.formicigenerans; |
| 82 | Akkermansia.muciniphila, unclassified.unclassified.S358, |
| | Bilophila.unclassified.S322, Dorea.formicigenerans; |
| 83 | Akkermansia.muciniphila, unclassified.unclassified.S358, |
| | Bacteroides.caccae, unclassified.unclassified.S306; |
| 84 | Akkermansia.muciniphila, unclassified.unclassified.S358, |
| | Dorea.formicigenerans, unclassified.unclassified.S306; |
| 85 | Akkermansia.muciniphila, Ruminococcaceae_UCG.002.unclassified.S91, |
| | Bacteroides.coprocola, unclassified.unclassified.S306; |
| 86 | Akkermansia.muciniphila, Ruminococcaceae_UCG.002.unclassified.S91, |
| | Bifidobacterium.longum, Dorea.formicigenerans; |
| 87 | Negativibacillus.unclassified.S269, Odoribacter.unclassified.S27, |
| | Oscillibacter.unclassified.S270, Bacteroides.unclassified.S176; |
| 88 | Christensenellaceae_R.7_group.unclassified.S209, Odoribacter.unclassified.S27, |
| | Oscillibacter.unclassified.S270, Bacteroides.unclassified.S176; |
| 89 | Akkermansia.unclassified.S361, Bifidobacterium.longum; |
| 90 | Akkermansia.muciniphila, Dorea.formicigenerans; |
| 91 | Akkermansia.muciniphila, unclassified.unclassified.S358, Bacteroides.fragilis, |
| | Sutterella.wadsworthensis, Negativibacillus.unclassified.S269, Bifidobacterium.longum, |
| | Bilophila.unclassified.S322, unclassified.unclassified.S306; |
| 92 | Akkermansia.unclassified.S361, Akkermansia.muciniphila, |
| | unclassified.unclassified.S358, Bacteroides.plebeius, Bifidobacterium.longum, |
| | Bilophila.unclassified.S322, Dorea.formicigenerans, unclassified.unclassified.S306; |
| 93 | Akkermansia.unclassified.S361, unclassified.unclassified.S358, Bacteroides.plebeius, |
| | Bacteroides.fragilis, Negativibacillus.unclassified.S269, Bacteroides.coprocola, |
| | Bacteroides.caccae, Alistipes.finegoldii; |
| 94 | Akkermansia.unclassified.S361, Ruminococcaceae_UCG.002.unclassified.S91, |
| | Bacteroides.plebeius, Bacteroides.fragilis, Bifidobacterium.longum, Bacteroides.caccae, |
| | Dorea.formicigenerans, Alistipes.finegoldii; |
| 95 | Akkermansia.unclassified.S361, unclassified.unclassified.S358, |
| | Ruminococcaceae_UCG.002.unclassified.S91, Bacteroides.plebeius, |
| | Bacteroides.coprocola, Bifidobacterium.longum, Bilophila.unclassified.S322, |
| | Bacteroides.caccae; |
| 96 | Akkermansia.muciniphila, unclassified.unclassified.S358, Bacteroides.plebeius, |
| | Bacteroides.fragilis, Bifidobacterium.longum, Dorea.formicigenerans, |
| | unclassified.unclassified.S306, Alistipes.finegoldii; |
| 97 | unclassified.unclassified.S358, Ruminococcaceae_UCG.002.unclassified.S91, |
| | Bacteroides.plebeius, Bacteroides.fragilis, Negativibacillus.unclassified.S269, |
| | Bacteroides.coprocola, Bilophila.unclassified.S322, Bacteroides.caccae; |
| 98 | Akkermansia.unclassified.S361, Bacteroides.plebeius, Bacteroides.fragilis, |
| | Sutterella.wadsworthensis, Bacteroides.coprocola, Bacteroides.caccae, |
| | Dorea.formicigenerans, unclassified.unclassified.S306; |
| 99 | Akkermansia.muciniphila, Ruminococcaceae_UCG.002.unclassified.S91, |
| | Bacteroides.plebeius, Bacteroides.fragilis, Bacteroides.coprocola, |
| | Bifidobacterium.longum, Bilophila.unclassified.S322, Dorea.formicigenerans; |
| 100 | Akkermansia.unclassified.S361, Akkermansia.muciniphila, |
| | unclassified.unclassified.S358, Ruminococcaceae_UCG.002.unclassified.S91, |
| | Negativibacillus.unclassified.S269, Bilophila.unclassified.S322, Bacteroides.caccae, |
| | unclassified.unclassified.S306; |
| 101 | Akkermansia.unclassified.S361, unclassified.unclassified.S358, |
| | Ruminococcaceae_UCG.002.unclassified.S91, Bacteroides.plebeius, |
| | Negativibacillus.unclassified.S269, Bacteroides.coprocola, Bifidobacterium.longum, |
| | unclassified.unclassified.S306 |
| 102 | Akkermansia.unclassified.S361, unclassified.unclassified.S358, |
| | Ruminococcaceae_UCG.002.unclassified.S91, Bacteroides.fragilis, |
| | Negativibacillus.unclassified.S269, Bacteroides.coprocola, Bilophila.unclassified.S322, |
| | Bacteroides.caccae; |
| 103 | Akkermansia.unclassified.S361, unclassified.unclassified.S358, |
| | Ruminococcaceae_UCG.002.unclassified.S91, Bacteroides.plebeius, |
| | Negativibacillus.unclassified.S269, Bilophila.unclassified.S322, Bacteroides.caccae, |
| | Dorea.formicigenerans; |
| 104 | Akkermansia.unclassified.S361, Akkermansia.muciniphila, Bacteroides.plebeius, |
| | Sutterella.wadsworthensis, Negativibacillus.unclassified.S269, |
| | Bilophila.unclassified.S322, Bacteroides.caccae, unclassified.unclassified.S306; |
| 105 | Akkermansia.unclassified.S361, Akkermansia.muciniphila, |
| | unclassified.unclassified.S358, Bacteroides.fragilis, Negativibacillus.unclassified.S269, |
| | Bacteroides.coprocola, Dorea.formicigenerans, unclassified.unclassified.S306; |
| 106 | Akkermansia.unclassified.S361, unclassified.unclassified.S358, |
| | Ruminococcaceae_UCG.002.unclassified.S91, Bacteroides.plebeius, |
| | Negativibacillus.unclassified.S269, Bilophila.unclassified.S322, Bacteroides.caccae, |
| | Dorea.formicigenerans; |
| 107 | Akkermansia.unclassified.S361, Ruminococcaceae_UCG.002.unclassified.S91, |
| | Bacteroides.plebeius, Bacteroides.fragilis, Negativibacillus.unclassified.S269, |
| | Bacteroides.coprocola, Bifidobacterium.longum, Bilophila.unclassified.S322; |
| 108 | Akkermansia.muciniphila, unclassified.unclassified.S358, |
| | Ruminococcaceae_UCG.002.unclassified.S91, Sutterella.wadsworthensis, |
| | Negativibacillus.unclassified.S269, Bifidobacterium.longum, Bacteroides.caccae, |
| | Dorea.formicigenerans; |
| 109 | Akkermansia.unclassified.S361, unclassified.unclassified.S358, Bacteroides.plebeius, |
| | Bacteroides.fragilis, Bilophila.unclassified.S322, Dorea.formicigenerans, |
| | unclassified.unclassified.S306, Alistipes.finegoldii; |
| 110 | Akkermansia.unclassified.S361, Akkermansia.muciniphila, |
| | unclassified.unclassified.S358, Bacteroides.plebeius, Negativibacillus.unclassified.S269, |
| | Bifidobacterium.longum, Bacteroides.caccae, Dorea.formicigenerans; |
| 111 | Akkermansia.unclassified.S361, Akkermansia.muciniphila, |
| | unclassified.unclassified.S358, Bacteroides.fragilis, Bacteroides.coprocola, |
| | Bifidobacterium.longum, Bilophila.unclassified.S322, unclassified.unclassified.S306; |
| 112 | Akkermansia.unclassified.S361, Akkermansia.muciniphila, |
| | unclassified.unclassified.S358, Bacteroides.plebeius, Negativibacillus.unclassified.S269, |
| | Bacteroides.caccae, Dorea.formicigenerans, unclassified.unclassified.S306; |
| 113 | Akkermansia.unclassified.S361, Akkermansia.muciniphila, Bacteroides.plebeius, |
| | Sutterella.wadsworthensis, Bacteroides.coprocola, Bacteroides.caccae, |
| | Dorea.formicigenerans, Alistipes.finegoldii; |
| 114 | Akkermansia.unclassified.S361, Ruminococcaceae_UCG.002.unclassified.S91, |
| | Bacteroides.plebeius, Sutterella.wadsworthensis, Bacteroides.coprocola, |
| | Bifidobacterium.longum, Bilophila.unclassified.S322, Bacteroides.caccae; |
| 115 | Akkermansia.muciniphila, unclassified.unclassified.S358, Bacteroides.plebeius, |
| | Sutterella.wadsworthensis, Bifidobacterium.longum, Bilophila.unclassified.S322, |
| | Bacteroides.caccae, Alistipes.finegoldii; |
| 116 | Christensenellaceae_R.7_group.unclassified.S209, Odoribacter.unclassified.S27, |
| | Ruminococcaceae_UCG.005.unclassified.S92, unclassified.unclassified.S136, |
| | Parabacteroides.merdae, Oscillibacter.unclassified.S270, Bacteroides.unclassified.S176, |
| | Parabacteroides.unclassified.S193; |
| 117 | Christensenellaceae_R.7_group.unclassified.S209, Odoribacter.unclassified.S27, |
| | Ruminococcaceae_UCG.005.unclassified.S92, unclassified.unclassified.S136, |
| | Parabacteroides.merdae, Oscillibacter.unclassified.S270, Bacteroides.unclassified.S176, |
| | Parabacteroides.unclassified.S193; |
| 118 | Family_XIII_UCG.001.unclassified.S64, |
| | Christensenellaceae_R.7_group.unclassified.S209, Acidaminococcus.unclassified.S307, |
| | Odoribacter.unclassified.S27, Parabacteroides.merdae, Oscillibacter.unclassified.S270, |
| | Bacteroides.unclassified.S176, Parabacteroides.unclassified.S193; |
| 119 | Bacteroides.fragilis, unclassified.unclassified.S136, Odoribacter.unclassified.S27, |
| | Acidaminococcus.unclassified.S307, Bacteroides.unclassified.S176, |
| | Bacteroides.coprocola, Bacteroides.finegoldii, Alistipes.finegoldii; |
| 120 | Akkermansia.unclassified.S361, Ruminococcaceae_UCG.002.unclassified.S91, |
| | unclassified.unclassified.S358, Sutterella.wadsworthensis, Bifidobacterium.longum, |
| | Parabacteroides.unclassified.S193, Bacteroides.finegoldii, |
| | unclassified.unclassified.S306; |
| 121 | Akkermansia.unclassified.S361, Ruminococcaceae_UCG.002.unclassified.S91, |
| | Bacteroides.plebeius, Ruminococcaceae_UCG.010.unclassified.S93, |
| | Bacteroides.unclassified.S176, Parabacteroides.unclassified.S193, |
| | Dorea.formicigenerans, Oscillibacter.unclassified.S270; |
| 122 | Akkermansia.unclassified.S361, Bacteroides.plebeius, |
| | Ruminococcaceae_UCG.005.unclassified.S92, Sutterella.wadsworthensis, |
| | Parabacteroides.unclassified.S193, Oscillibacter.unclassified.S270, |
| | Bilophila.unclassified.S322, Negativibacillus.unclassified.S269; |
| 123 | Akkermansia.muciniphila, Christensenellaceae_R.7_group.unclassified.S209, |
| | Ruminococcaceae_UCG.005.unclassified.S92, unclassified.unclassified.S358, |
| | Parabacteroides.merdae, Bacteroides.coprocola, Oscillibacter.unclassified.S270, |
| | Negativibacillus.unclassified.S269; |
| 124 | Akkermansia.muciniphila, Bacteroides.plebeius, Odoribacter.unclassified.S27, |
| | Negativibacillus.unclassified.S269, Parabacteroides.merdae, Bifidobacterium.longum, |
| | Alistipes.finegoldii, Negativibacillus.unclassified.S269; |
| 125 | Ruminococcaceae_UCG.002.unclassified.S91, Bacteroides.plebeius, |
| | Odoribacter.unclassified.S27, Ruminococcaceae_UCG.010.unclassified.S93, |
| | Bacteroides.unclassified.S176, Bifidobacterium.longum, Dorea.formicigenerans, |
| | Alistipes.finegoldii; |
| 126 | Akkermansia.unclassified.S361, Christensenellaceae_R.7_group.unclassified.S209, |
| | Odoribacter.unclassified.S27, Ruminococcaceae_UCG.010.unclassified.S93, |
| | Parabacteroides.merdae, Parabacteroides.unclassified.S193, Bacteroides.coprocola, |
| | Bilophila.unclassified.S322; |
| 127 | Akkermansia.unclassified.S361, Ruminococcaceae_UCG.002.unclassified.S91, |
| | Odoribacter.unclassified.S27, Ruminococcaceae_UCG.010.unclassified.S93, |
| | Bacteroides.unclassified.S176, Bacteroides.caccae, Parabacteroides.merdae, |
| | Dorea.formicigenerans; |
| 128 | Akkermansia.muciniphila, Akkermansia.unclassified.S361, Sutterella.wadsworthensis, |
| | Family_XIII_UCG.001.unclassified.S64, Bacteroides.unclassified.S176, |
| | Bacteroides.caccae, Parabacteroides.merdae, Alistipes.finegoldii. |

In the above groups, the first half of the taxa are those to be determined in the first phase, and the second half, the bacterial taxa to be determined in the second phase.

In a more preferred embodiment, the taxa is selected from the group consisting of Akkermansia spp., Akkermansia muciniphila, Bacteroides fragilis, Bacteroides plebeius, Negativibacillus spp., Bacteroides coprocola, Bacteroides caccae, and Dorea formicigenerans.

In an even more preferred embodiment, in the first phase of the method the levels of Akkermansia spp., Akkermansia muciniphila, Bacteroides fragilis and Bacteroides plebeius are determined to classify the subject to have CRC, and in the second phase the levels of Negativibacillus spp., Bacteroides coprocola, Bacteroides caccae and Dorea formicigenerans are determined to classify a subject to have a risk of developing CRC. Preferably, in the first phase higher levels of Akkermansia spp. and/or Akkermansia muciniphila and lower levels of Bacteroides fragilis and/or Bacteroides plebeius are associated with CRC, and in the second phase higher levels of Negativibacillus spp. and/or Bacteroides coprocola and/or lower levels of Bacteroides caccae and/or Dorea formicigenerans are associated with a risk of developing CRC.

In the most preferred embodiment, the bacteria combinations whose levels are determined are Akkermansia.unclassified.S361 and Akkermansia.muciniphila for step (ii) (phase 1) and, Bacteroides.coprocola and Dorea.formicigenerans for step (iii) (phase 2).

In a preferred embodiment, in the first and second phase, if a first ratio comprising the centered-log ratios (clr) of the following taxa:

$$\frac{Akkermansia spp . + Akkermansia muciniphila}{Bacteroides fragilis + Bacteroides plebeius}$$

is higher than-0.5512273; and a second ratio

$$\frac{Bacteroides coprocola + Negativibacillus spp .}{Dorea formicigenerans + Bacteroides caccae}$$

is higher than 0, the subject is diagnosed to have a risk of developing CRC.

In another embodiment of the first aspect of the invention, when the sample is FIT negative, the bacterial taxa are selected from the group consisting of: Alistipes.putredinis, Anaerostipes.hadrus, Bacteroides.coprocola, Bacteroides.eggerthii, Bifidobacterium.animalis, Bifidobacterium.bifidum, Bifidobacterium.longum, Blautia.massiliensis, Blautia.obeum, Coprococcus.comes, Coprococcus.eutactus, Dorea.longicatena, Fusobacterium.necrophorum, Parvimonas.micra, Peptostreptococcus.stomatis, Solobacterium.moorei, Bifidobacterium.unclassified.S5, Adlercreutzia.unclassified.S168, Porphyromonas.unclassified.S30, Paraprevotella.unclassified.S182, Prevotella.unclassified.S33, Parvimonas.unclassified.S67, Coprococcus.unclassified.S223, Dorea.unclassified.S225, Eisenbergiella.unclassified.S226, Lachnoclostridium.unclassified.S77, Peptostreptococcus.unclassified.S87, Peptococcus.unclassified.S249, Flavonifractor.unclassified.S265, GCA.900066225.unclassified.S267, Negativibacillus.unclassified.S269, Oscillospira.unclassified.S271, Ruminococcaceae_UCG.008.unclassified.S281, Erysipelotrichaceae_UCG.003.unclassified.S297, Faecalitalea.unclassified.S300, unclassified.unclassified.S306; Acidaminococcus.unclassified.S307, Fusobacterium.unclassified.S106, Desulfovibrio.unclassified.S323, Blautia.stercoris, Butyrivibrio.crossotus, Parabacteroides.distasonis, Roseburia.inulinivorans, Sellimonas.intestinalis, Olsenella.unclassified.S24, Odoribacter.unclassified.S27, Weissella.unclassified.S204, Streptococcus.unclassified.S55, Christensenellaceae_R.7_group.unclassified.S209, Lachnospiraceae UCG.010.unclassified.S242, Marvinbryantia.unclassified.S244, Intestinibacter.unclassified.S818, Ruminococcaceae_NK4A214_group.unclassified.S277, Ruminococcaceae_UCG.005.unclassified.S92, Ruminococcaceae_UCG.014.unclassified.S94, Veillonella.unclassified.S104 and Akkermansia.unclassified.S361.

In a preferred embodiment, the taxa is selected from the group consisting of Fusobacterium.unclassified.S106, Peptostreptococcus.unclassified.S87, Erysipelotrichaceae_UCG.003.unclassified.S297, Alistipes.putredinis, Prevotella.unclassified.S33, Akkermansia.unclassified.S361, Coprococcus.comes, Bifidobacterium.longum. Preferably, the combination of any of these taxa classifies a subject to have a risk of developing CRC.

In a more preferred embodiment, in the first phase levels Fusobacterium.unclassified.S106, Peptostreptococcus.unclassified.S87, Erysipelotrichaceae_UCG.003.unclassified.S297 and Alistipes.putredinis; and in the second phase levels of Prevotella.unclassified.S33, Akkermansia.unclassified.S361, Coprococcus.comes and Bifidobacterium.longum; are determined to classify a subject to have a risk of developing CRC.

In another preferred embodiment of the first aspect of the invention, a subject classified in a cohort of subjects as having risk of developing CRC in step (iii) is considered to require a colonoscopy, and those subjects not classified in a cohort of subjects as having risk of developing CRC in step (iii) are considered to not require a colonoscopy.

The computer algorithm of step (iii) in the method of the present disclosure is selected from the group consisting of an artificial intelligence algorithm, a machine learning algorithm, and a trained neural network algorithm. Preferably, the computer algorithm is a trained neural network algorithm.

In a second aspect, the present invention relates to a kit comprising:
(a) reagents for conducting a method for determining the presence or abundance of the bacteria in a fecal sample to determine the levels of two or more bacterial taxa in step (i) of the method of the previous embodiments; and
(b) a computer program comprising instructions which, when the program is executed by a computer, cause the computer to carry out steps (ii) and (iii) of the inventive method.

In a preferred embodiment, in step (a) is determined the levels of three or more taxa in the step (i) of the method.

In a preferred embodiment, the kit comprises:
(a) reagents for conducting a method for determining the presence or the abundance of the bacteria in a fecal sample to determine the levels of two or more bacterial taxa in step (i) of the method of second aspect of the invention; and
(b) a computer program stored on a computer-readable data carrier or chip, comprising instructions which, when the program is executed by a computer, cause the computer to carry out steps (ii) and (iii) of the method of the invention.

In a preferred embodiment, in step (a) is determined the levels of three or more taxa in the step (i) of the method.

In a more preferred embodiment, the reagents are for conducting 16S rRNA gene sequencing.

### Examples

The examples given below are for illustrative purposes only and do not limit the invention described above in any way.

### Example 1: Sample Collection and Subjects

A total of 2,889 FIT-positive (>20 μg hemoglobin/g feces) and 246 FIT-negative (<20 μg hemoglobin/g feces) samples from the Catalan CRC Screening Program were analysed. summary of the distribution of FIT-positive samples across several characteristics is shown in TABLE 1.

TABLE 1
CHARACTERISTICS OF THE INCLUDED INDIVIDUALS.

| | % Presence | Individuals | | Median | Clinical relevance | |
|---|---|---|---|---|---|---|
| Sex | of polyps* | N | % | Age* | N | % |
| All | 66.82 | 2,889 | 100 | 60 | 1193 | 41.29 |
| Males | 74.62 | 1548 | 53.58 | 60 | 742 | 47.93 |
| Females | 57.87 | 1341 | 46.42 | 61 | 451 | 33.63 |

*SAMPLES WITH ‘NA’ VALUE FOR THIS PARAMETER ARE EXCLUDED FROM THE CALCULATION.

Collected metadata comprised six different clinical variables for each sample, including the diagnosis after colonoscopy evaluation (TABLE 2), the number of polyps, the FIT value (μg of hemoglobin/g of feces), the hospital at which the sample was collected, and the donor's sex and age. The considered colonoscopy diagnoses were: Negative (N), colorectal cancer (CRC) and different lesions that can be relevant in the colorectal cancer development: Carcinoma in situ (CIS), high risk lesion (HRL), intermediate risk lesion (IRL), low risk lesion (LRL) and lesion not associated to risk (LNAR) (23). Additionally, the samples were classified into two groups according to the clinical relevance of the colonoscopy-based diagnosis (24). CRC, CIS, HRL and IRL were considered clinically relevant colonoscopy (CR) and N, LNAR and LRL as non-clinically relevant colonoscopy (Non-CR).

TABLE 2
CRITERIA AND DISTRIBUTION OF THE COLONOSCOPY-BASED DIAGNOSIS TYPES.
COLUMNS INDICATE, IN THIS ORDER, THE DIAGNOSIS GROUP, THE CRITERIA
FOR CLASSIFICATION IN THE GROUP, THE NUMBER OF SAMPLES OF THIS
STUDY IN THE GIVEN GROUP, AND THE CLINICAL RELEVANCE.

| | | FIT | FIT | |
|---|---|---|---|---|
| | | Positive | Negative | Clinical |
| Diagnosis group | Criteria | (N) | (N) | relevance |
| Negative (N) | Absence of adenomas or polyps | 925 | 81 | Non-CR |
| Lesion Not Associated | <20 hyperplastic polyps <10 mm limited to rectal | 90 | 0 | Non-CR |
| to Risk (LNAR) | or sigma | | | |
| Low Risk Lesion | 1-2 tubular adenomas <10 with low-grade | 681 | 49 | Non-CR |
| (LRL) | dysplasia or 1-2 serrated polyps <10 mm without | | | |
| | dysplasia | | | |
| Intermediate Risk | 3-4 tubular adenomas <10 mm with low-grade | 638 | 28 | CR |
| Lesion (IRL) | dysplasia or | | | |
| | 1-4 tubular adenomas 10-19 mm with low-grade | | | |
| | dysplasia or | | | |
| | 1-4 adenomas <20 mm with villous component | | | |
| | and/or high-grade dysplasia (intraepithelial | | | |
| | carcinoma) and/or intramucosal carcinoma, or | | | |
| | 3-4 serrated polyps <10 mm without dysplasia, or | | | |
| | 1-4 serrated polyps 10-19 mm without dysplasia, or | | | |
| | 1-4 serrated polyps <20 mm with dysplasia. | | | |
| High Risk Lesion | >=5 Adenomas/ Serrated polyps, or | 397 | 37 | CR |
| (HRL) | >=1 Adenomas/ Serrated polyps >=20 mm | | | |
| Carcinoma in situ (CIS) | Noninvasive, intramucosal carcinoma.Stage 0. | 24 | 0 | CR |
| Colorectal cancer | Invasive colorectal adenocarcinoma. From Stage I | 134 | 51 | CR |
| (CRC) | to IV. | | | |

### Example 2: DNA Extraction and 16S RRNA Sequencing

In the following, 16S rRNA gene sequencing was used as a method for identification, classification and quantitation of bacterial taxa within complex biological mixtures such as fecal samples. However, the skilled artisan appreciates that also other analytical methods can be used if suitable or desired, e.g., the polymerase chain reaction (PCR), PCR multiplexing, “next generation sequencing” (NGS), RNA panels, proteomics, gaschromatography/mass spectrometry, and liquid chromatography/masspectrometry.

Aliquots of 500 μl from FIT samples were prepared in a test tube and stored at −80° C. until further processing. DNA was extracted using the DNeasy PowerLyzer PowerSoil Kit (Qiagen, ref. QIA12855) following manufacturer's instructions. The extraction tubes were agitated twice in a 96-well plate using Tissue lyser II (Qiagen) at 30 Hz/s for 5 min. 4 μl of each DNA sample were used to amplify the V3-V4 regions of the bacterial 16S ribosomal RNA gene, using the following universal primers in a limited cycle PCR:

V3-V4-Forward
(5′-TCGTCGGCAGCGTCAGATGTGTATAAGAGACAGCCTACGGGNGGCWGCAG-3′)
and
V3-V4-Reverse
(5′-GTCTCGTGGGCTCGGAGATGTGTATAAGAGACAGGACTACHVGGGTATCTAATCC-3′).

To prevent unbalanced base composition in further MiSeq sequencing, sequencing phases were shifted by adding a variable number of bases (from 0 to 3) as spacers to both forward and reverse primers (a total of 4 forward and 4 reverse primers were used). The PCR was performed in 10 μl volume reactions with 0.2 μM primer concentration and using the Kapa HiFi HotStart Ready Mix (Roche, ref. KK2602). Cycling conditions were initial denaturation of 3 min at 95° C. followed by 25 cycles of 95° C. for 30 s, 55° C. for 30 s, and 72° C. for 30 s, ending with a final elongation step of 5 min at 72° C.

After the first PCR step, water was added to a total volume of 50 μl and reactions were purified using AMPure XP beads (Beckman Coulter) with a 0.9× ratio according to manufacturer's instructions. PCR products were eluted from the magnetic beads with 32 μl of Buffer EB (Qiagen) and 30 μl of the eluate were transferred to a fresh 96-well plate. The primers used in the first PCR contained overhangs allowing the addition of full-length Nextera adapters with barcodes for multiplex sequencing in a second PCR step, resulting in sequencing ready libraries. To do so, 5 μl of the first amplification was used as template for the second PCR with Nextera XT v2 adaptor primers in a final volume of 50 μl using the same PCR mix and thermal profile as for the first PCR but only 8 cycles. After the second PCR, 25 μl of the final product was used for purification and normalization with SequalPrep normalization kit (Invitrogen), according to the manufacturer's protocol. Libraries were eluted in 20 μl and pooled for sequencing.

Final pools were quantified by qPCR using Kapa library quantification kit for Illumina Platforms (Kapa Biosystems) on an ABI 7900HT real-time cycler (Applied Biosystems). Sequencing was performed in Illumina MiSeq with 2×300 bp reads using v3 chemistry with a loading concentration of 18 pM. To increase the diversity of the sequences 10% of PhIX control libraries were spiked in.

Two bacterial mock communities were obtained from the BEI Resources of the Human Microbiome Project (HM-276D and HM-277D), each containing genomic DNA of ribosomal operons from 20 bacterial species (25). Mock DNAs were amplified and sequenced in the same manner as all other FIT samples. Negative controls of the DNA extraction and PCR amplification steps were also included in parallel, using the same conditions and reagents. These negative controls provided no visible band or quantifiable DNA amounts by Bioanalyzer, whereas all of our samples provided clearly visible bands after 25 cycles.

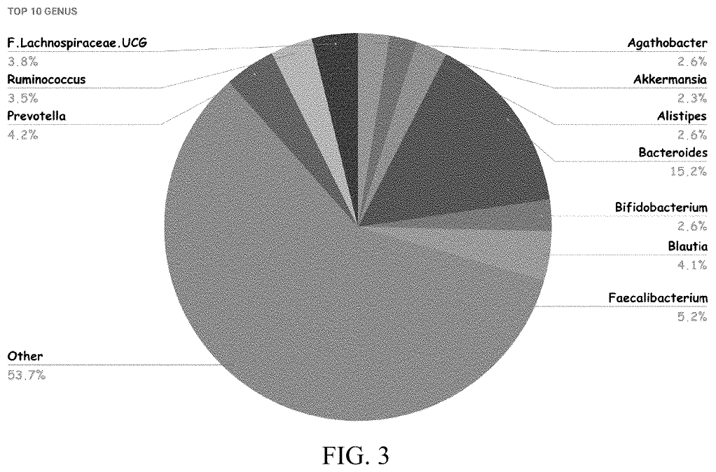

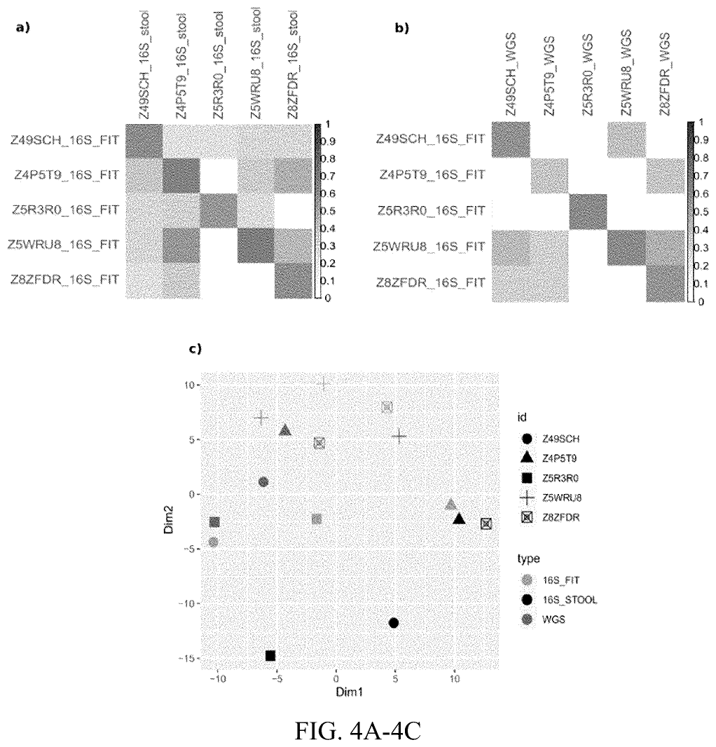

For the FIT positive group, it was obtained a mean value of 56,219.03 filtered reads per sample, which comprised a total of 376 assigned taxa. Bacteroidetes and Firmicutes were the most represented phyla, and the ten most abundant genera were, in this order: Bacteroides, Faecalibacterium, Prevotella, Blautia, F. Lachnospiraceae.UCG, Ruminococcus, Agathobacter, Bifidobacterium, Alistipes and Akkermansia (FIG. 3). These results are consistent with previous studies using stool samples (49-53).Similarity of microbiome profiles obtained from FIT and fecal samples was also confirmed by comparing data from five individuals included in this study for which fecal whole genome shotgun Illumina data and Ion-Torrent V2-4, V6-8 16S profiling data were available (35) (FIG. 4).

### Example 3. Microbiome Analysis

The dada2 (v. 1.10.1) pipeline (27) was used to obtain an amplicon sequence variants (ASV) table for each of the sequencing runs separately. The quality profiles of forward and reverse sequencing reads were examined using the plotQualityProfile function of dada2 and, according to these plots, low-quality sequencing reads were filtered and trimmed using the filterAndTrim function. A matrix with learned error rates was obtained with the learnErrors dada2 function.

Dereplication (combining identical sequencing reads into unique sequences) was performed, sample inference (from the matrix of estimated learning error rates) and merged paired reads to obtain full denoised sequences. From these, chimeric sequences were removed. Taxonomy was assigned to ASVs by mapping to the SILVA 16s rRNA database (v. 132) (28). Negative controls (non-template samples) and positive controls (mock microbial communities comprising a mixture of 20 strains with known proportions) were sequenced and analyzed in each of the runs to assess the possible contamination background and evaluate the accuracy of the pipeline. ASV and Taxonomy tables were obtained for each run separately, and then, merged the results. Samples without metadata information and the controls were discarded in further analyses.

A phylogenetic tree was reconstructed by using the phangorn (v. 2.5.5) (29) and Decipher R packages (v 2.10.2) (30) and integrated it with the merged ASV and Taxonomy tables and their assigned metadata creating a phyloseq (v. 1.26.1) object (31). It was characterized alpha diversity metrics including Observed index, Shannon, Simpson, InvSimpson, PD Chao1, ACE and also standard error measures such as se.Chaol and se.ACE using the estimate_richness function of the phyloseq package. Using the picante package (v. 1.8.1), it was computed Faith's phylogenetic diversity, an alpha diversity metric that incorporates branch lengths of the phylogenetic tree.

Additionally, it was calculated different distance metrics based on the differences in taxonomic composition between samples using the Phyloseq and Vegan (v. 2.5-6) packages (Oksanen et al. 2019, Vegan: Community Ecology Package. https://CRAN.R-project.org/package=vegan). These metrics include Jensen-Shannon Divergence (JSD), Weighted-Unifrac, Unweighted-unifrac, Bray-Curtis dissimilarity, Jaccard and Canberra. It was also computed Aitchison distances between samples using the cmultRepl and codaSeq.clr functions from the CodaSeq (v. 0.99.6) (32) and zCompositions (v. 1.3.4) (33) packages. Normalization was performed by transforming counts to centered log-ratios (clr) (34). The centered log-ratio is a transformation of the raw counts to make the samples comparable, considering the compositional nature of the microbiome data. It is the application of log to the ratio of the observed frequencies and their geometric mean. Prior to this transformation, a multiplicative simple zero replacement as implemented in cmultRepl function of the zCompositions package (Indicating method=“CZM”) was done. The clr can result in both positive and negative values. Samples with fewer than 1000 reads and taxa that appeared in few samples and low abundances were filtered out. Finally, taxa at each taxonomic rank was agglomerated to study trends at different taxonomic depths.

### Example 4. Statistical Analysis

Associations between clinical variables and the overall microbial composition of the samples were assessed by performing Permutational Multivariate Analysis of Variance (PERMANOVA) using the adonis function from the Vegan R package (v. 2.5-6) with the seven-distance metrics mentioned above. Diagnosis, sex and age variables were considered as covariates. We also applied the Analysis of similarities (ANOSIM) test by using anosim function from the Vegan R package to assess differences between and within groups.

It was performed a differential abundance analysis using clr data for the different taxonomic ranks across various clinical variables using linear models implemented in the R package lme4 (v. 1.1-21) (41). A linear model was built, including Diagnosis (Dx), sex, age, number of polyps and hospital and FIT value (only for FIT positive samples) as fixed effects, and the sequencing run as a random effect to account for possible batch effects. This linear model was evaluated considering all the diagnoses, but also making a comparison of CRC versus non-CRC samples by changing all other diagnoses to “Others”. A second linear model was applied that considered as fixed effect a variable called Risk instead of the Diagnosis in order to assess the differences between samples with CR or Non-CR colonoscopy, as defined above (TABLE 2).

An Analysis of Variance (ANOVA) was applied to assess the significance for each of the fixed effects included in the models using the Car R package (v. 3.0-6) (42). To assess particular differences between groups, a multiple comparisons was performed to the results obtained in the linear models using the Tukey test in the function glht from multcomp R package (v. 1.4-12) (43). It was applied Bonferroni as a multiple testing correction, and statistical significance was defined at p values lower than 0.05. In addition, it was used the selbal package (v. 0.1.0) (44) to study groups of taxa (balances) with potential predictive power for CRC status in FIT positive samples.

### Example 5. Machine Learning Classification

A further aspect of the present invention relates to a novel two-phase classifier, which magnifies the inclusion of colorectal cancer and clinically relevant cases and prioritizes the reduction of false negatives instead of false positives. Feature selection is based on a differential analysis: combination of centered log ratios (Clr) of the selected taxa with clinical variables (sex, age and hemoglobin content).

In brief, it was developed a predictive model based on a two-phase classification (FIG. 2) using a neural network (NN) algorithm implemented in the caret package (v.6.0-85) (47). For each phase it was trained a random 75% of the data with a 10-fold cross validation and tested with the remaining samples. The process was repeated 100 times to avoid “lucky” splits and to evaluate the variability in predictive performance. A feature selection was performed based on the differential abundance results including taxa found as having significantly different abundances in our invention and incorporating hemoglobin content, age and sex variables. Samples with missing values for the considered metadata were removed. Taxa abundances were included as clr. The two-phase classifier proceeds as follows: in the first phase the method classifies CRC vs non-CRC samples. Samples that are classified as non-CRC in the first phase, including misclassified CRCs in order to improve the sensitivity are subjected to a second model that classifies CR vs non-CR samples. At the end of the two-phase classification the mean percentage of misclassified CRC and CR samples was calculated, and the performance of the model was evaluated.

To validate this strategy a model trained with all the CRIPREV samples was built, and tested it in two independent datasets: a cohort from the USA (48) and 100 extra samples from the same Catalan screening. For the USA cohort, it was applied the Catalan hemoglobin threshold (>20 μg of hemoglobin/g of feces) to select the FIT-positive samples to include in the validation. It was processed their raw data following exactly the same methodology as disclosed in the present document (See Microbiome analysis, Materials and methods). It was unfortunately not assigned Bacteroides fragilis, likely because that study only used the V4 region of the 16S rRNA gene as compared to V3-V4 in the present invention. In the following, the design and building of the classifier is described in more detail.

### Example 6. Design and Building of the Classifier on Fit Positive Samples

The two-phase classifier proceeds as follows: in the first phase the method classifies colorectal cancer (CRC) vs non-CRC samples. Samples that are classified as non-CRC in the first phase are subjected to a second model that classifies Clinically relevant (CR) vs non-CR samples. Clinically relevant is a grouping of colonoscopy diagnoses ranging from mid-risk lesions to high-risk lesions and CRC that require clinical follow up.

The input used by the model is a three data associated with the FIT (Sex, Age, and FIT Value) and a normalized and filtered Amplicon Sequence Variant (ASV) table (obtained from the sequencing data as explained herein). The ASV is limited to a set of selected taxa (optimal model, in terms of inclusion of CR cases, included 4 taxa as explained in the herein, but could be other combinations from the relevant taxa identified in the present specification).

The model was trained using ˜2800 sample data from the CRIPREV project. For the first phase classification a 10-fold cross validation was made, and the best model was the one used to predict the independent test set. Some of the specificities of the model:

Method: nnet, implemented in the caret package by using the train function (v. 6.0-85).

MaxNWts (The maximum allowable number of weights): 2000.

Weights: we change the weights, penalizing more the expected minor class: 0.75 for CRC and 0.25 for others.

After the prediction, a confusion matrix is constructed and samples that are classified as others are subjected to a second classification detailed below. If the model classified all the samples to CRC (AUC: 0.5, nul ability to classify) all the samples are subjected to the second classification.

For the second phase, the same training set was used, but CRC samples were removed from this training set and the mid-risk and high-risk lesions were labeled as clinically relevant. The model was trained to recognize the clinically relevant samples. For this, a 10-fold cross validation was made, and the best model was the one used to predict the independent test set. Some of the specificities of the model:

Method: nnet, implemented in the caret package (v. 6.0-85).

MaxNWts (The maximum allowable number of weights): 2000.

Weights: we change the weights, penalizing more the expected minor class: 0.60 for Clinically relevant samples and 0.40 for non-clinically relevant samples.

### Performance Evaluation

To evaluate the strategy, three independent strategies were applied:

### 1 CRIPREV Samples

For each phase it was constructed a model training a random 75% of the data with a 10-fold cross validation and tested with the remaining samples. The process was repeated 100 times to avoid “lucky” splits and to evaluate the variability in predictive performance. It was performed a feature selection based on the differential abundance results including taxa found as having significantly different abundances in our invention and incorporating FIT-value, age and sex variables. Samples with missing values for the considered metadata were removed.

### 2. Independent Study

It was trained the model with 100% of the CRIPREV data and tested the performance on an independent dataset cohort from the USA, of 135 samples, from a previous published study. For this last study it was applied the Catalan hemoglobin threshold (>20 μg of hemoglobin/g of feces) to select the FIT-positive samples to include in the validation. Their raw data was processed following exactly the same methodology as described in the present document. Unfortunately, Bacteroides fragilis could not be assigned, likely because that study only used the V4 region of the 16S rRNA gene as compared to V3-V4 in the invention.

3. Newly Obtained Samples from the Catalan Screening Program

Using the model trained with 100% of the CRIPREV data, the performance on an independent dataset of 100 further FIT positive samples from the Catalan CRC screening was tested. No-limiting examples for threshold values helpful for diagnosing CRC using the inventive method are described in the following.

Different thresholds were assessed considering:
(I) A ratio calculated from all the dysregulated taxa (overrepresented taxa/underrepresented taxa) for each of the phases.
(II) A ratio calculated from a 4 taxa panel (overrepresented taxa/underrepresented taxa) for each phase.
(III) Means of the key species in clinically relevant groups.

The best result so far was considering the second option, a filter based on a threshold using two different ratios including a 4 taxa panel for each phase.

From the amplicon sequence variant table normalized by centered log-ratios (clr) two ratios were computed:

### First Ratio:

$$\frac{Akkermansia unclassified . S 361 + Akkermansia muciniphila}{Bacteroides fragilis + Bacteroides plebeius}$$

### Second Ratio:

$$\frac{Bacteroides coprocola + Negativibacillus unclassified . S 269}{Dorea formicigenerans + Bacteroides caccae}$$

It was applied a filter with the condition of having the first ratio higher than-0.5512273 (based on the mean of the first ratio in CRC patients) or the second ratio higher than 0.

The results obtained were:

### Using the CRIPREV Dataset:

Percentage of detected Clinically relevant samples: 85.41.
Percentage of CRC samples: 86.57.
Percentage of saved colonoscopies: 14.92.

### Using the Validation Dataset:

Percentage of detected Clinically relevant samples: 81.25.
Percentage of CRC samples: 88.
Percentage of saved colonoscopies: 14.

### Alpha and Beta Diversity

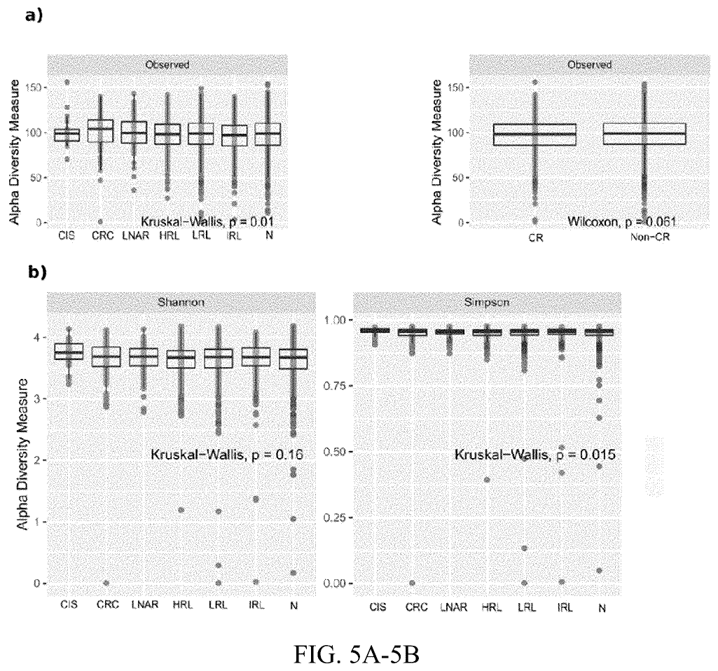

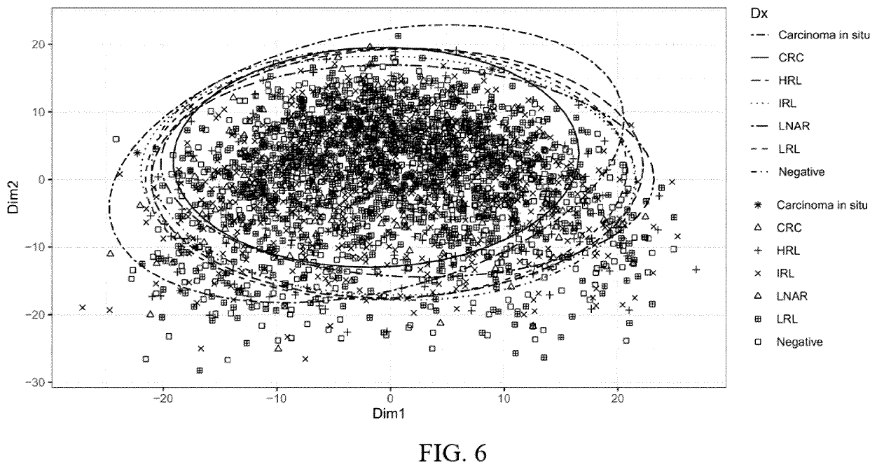

It was quantified the overall diversity of the microbiome in the samples by computing alpha and beta diversity metrics. It was observed significant differences (P<0.05) in the observed index alpha diversity metric (which measures the number of species per sample) when considering all diagnoses but not when specifically comparing CR vs Non-CR samples (FIG. 5). For the Shannon and Simpson indices, which consider differences in abundance, it was only observed significant differences with the Simpson index (which assigns more weight to dominant species) when considering all diagnoses.

It was produced MDS plots using distances between the microbial profiles of samples (beta diversity) such as the Aitchison distance (FIG. 5). It was not observed any clear clustering of samples with the same diagnosis or risk (CR vs non-CR). However, with the adonis test and Aitchison distance, it was detected a significant effect of the diagnosis (P=0.001) considering sex and age as covariates, and the sequencing run as a possible source of batch effect. The ANOSIM test also supported significant but subtle differences between the diagnostic groups and a higher similarity within groups (R: 0.07463, p-value: 0.001). Altogether, this suggests the existence of significant but subtle differences in the overall microbiome composition between FIT-positive samples with different colonoscopy outcomes.

### Example 7: Microbiome in Fit Positive Samples

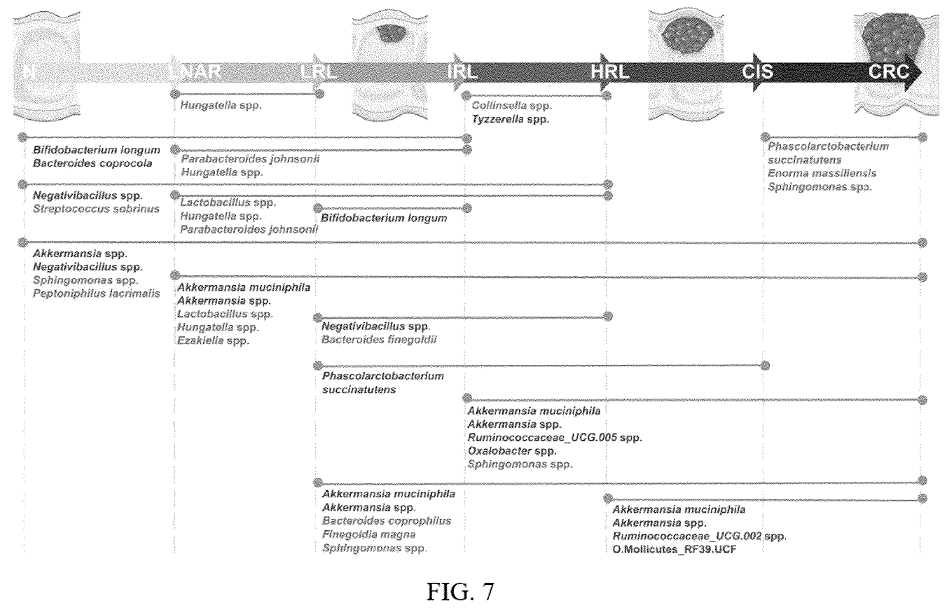

Using comparative analysis, significant differences were detected in the relative abundance of several taxa according to the various fixed effect variables (FIT positive samples). These analyses identified, for instance, 34 species whose abundance changed significantly across colonoscopy diagnosis (Table 3 and FIG. 7).

TABLE 3
SUMMARY OF THE DIFFERENTIAL ABUNDANCE ANALYSIS
RESULTS CONSIDERING ALL THE DIAGNOSES FOLLOWING
THE PATH FROM HEALTHY COLON TO COLORECTAL CANCER.
USED LINEAR MODEL: TAX_ELEMENT~DIAGNOSIS +
HOSPITAL + SEX + AGE + N_POLYPS + FIT_VALUE + (1|RUN).

| | Phylum | Class | Order | Family | Genus | Species |
|---|---|---|---|---|---|---|
| Diagnosis | 3 | 4 | 6 | 10 | 18 | 34 |
| Hospital | 6 | 10 | 14 | 27 | 73 | 112 |
| Sex | 4 | 7 | 13 | 30 | 96 | 132 |
| Age | 2 | 3 | 7 | 15 | 42 | 78 |
| N_polyps | 1 | 2 | 2 | 4 | 15 | 33 |
| FIT_value | 1 | 1 | 1 | 4 | 10 | 14 |

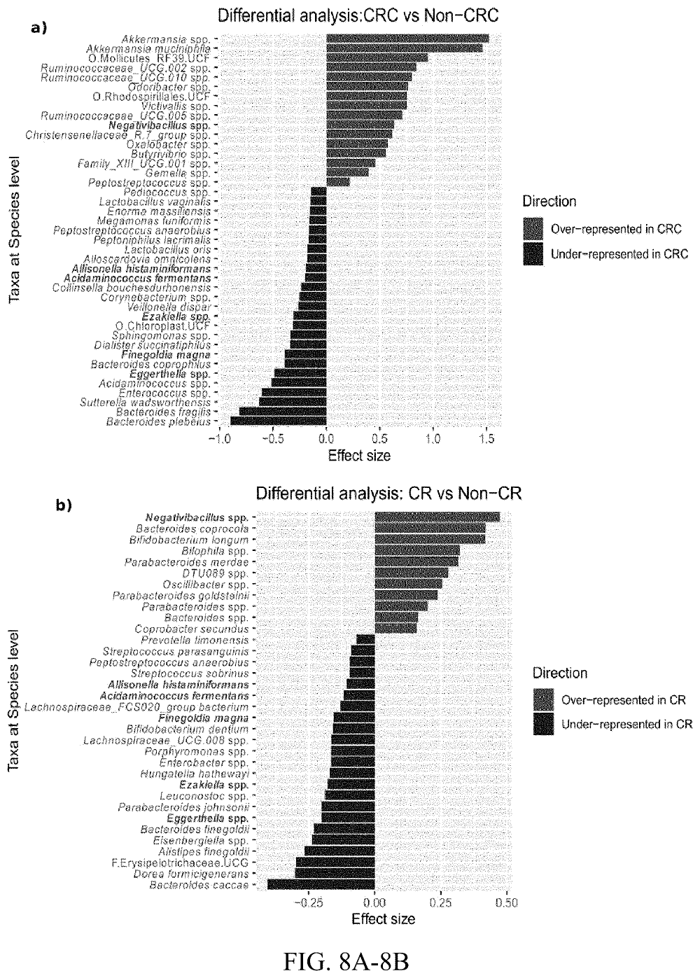

Based on the observation that CRC was the most distinct diagnosis (FIG. 7), it was specifically compared CRC to non-CRC samples, which revealed 41 differentially abundant species (FIG. 8A). These included overrepresentation of Akkermansia muciniphila and Akkermansia spp., as well as underrepresentation of Bacteroides plebeius and Bacteroides fragilis in CRC compared to non-CRC samples. In addition, using the selbal package for the same comparison (CRC vs non-CRC), it was identified that the ratio between species (balance) most associated with CRC-status was given by a decreased ratio (as compared to non-CRC samples) between a group of taxa comprising B. fragilis (G1: Bifidobacterium spp., Bacteroides fragilis, Sutterella wadsworthensis, and Eggerthella spp.), with respect to a second group of taxa including Akkermansia spp. (G2: Akkermansia spp., Gemella spp., Peptostreptococcus stomatis, Adlercreutzia spp. and Butyrivibrio spp.). Finally, it was applied the same linear model to the comparison of CR vs Non-CR samples, which identified 34 differentially abundant species (FIG. 8B).

Colorectal polyps, which are benign tumors that project onto the colon mucus and protrude into intestinal lumen (54), have long been identified as potential precursors of CRC. The present disclosure includes 66.82% samples for which colonoscopy detected the presence of polyps, with numbers of polyps ranging from 1 to 22. It was observed that some CRC samples had no polyps, whereas some negative samples had from 1 to 3 polyps, and some lesions that were not associated with a clinically relevant colonoscopy had a considerable amount of polyps (from 1 to 11 polyps).Species whose abundance correlated significantly with the number of polyps were detected (TABLE 4).

TABLE 4
TABLE OF SPECIES FOUND AS DIFFERENTIALLY
ABUNDANT ACCORDING TO THE NUMBER OF POLYPS,
AND THE SIGNIFICANCE VALUES (P-VALUE).

| Species | P-value |
|---|---|
| Bacteroides vulgatus | 0.03057510581 |
| Holdemanella spp. | 0.03644160564 |
| Phascolarctobacterium faecium | 0.02403576075 |
| Bacteroides caccae | 0.001737505877 |
| Blautia massiliensis | 0.04195995777 |
| Lachnospira spp. | 0.01032635218 |
| Dorea formicigenerans | 0.02794392414 |
| Lachnospiraceae_ND3007_group spp. | 0.01079273313 |
| Odoribacter splanchnicus | 0.04758839409 |
| Bacteroides clarus | 0.02370482911 |
| Christensenellaceae_R.7_group spp. | 0.03718813875 |
| Ruminococcaceae_UCG.005 spp. | 0.01925587147 |
| Parabacteroides goldsteinii | 0.03205297485 |
| Streptococcus sobrinus | 0.0002100715512 |
| Negativibacillus spp. | 0.007849255824 |
| Bifidobacterium angulatum | 0.03089555766 |
| Eggerthella lenta | 0.03397138805 |
| Intestinimonas spp. | 0.03928919384 |
| Haemophilus parainfluenzae | 0.01846211223 |
| unclassified Kingdom | 0.02109477726 |
| Enorma massiliensis | 0.01914417619 |
| Lactobacillus reuteri | 0.02261353835 |
| Fournierella spp. | 0.03945379814 |
| Ruminococcaceae_UCG.010 spp. | 0.0126820256 |
| GCA.900066575 spp. | 0.01694946601 |
| Solobacterium spp. | 0.03291648201 |
| DNF00809 spp. | 0.01976058036 |
| Collinsella bouchesdurhonensis | 0.005164515906 |
| Allisonella spp. | 0.01588205507 |
| GCA.900066225 spp. | 0.02704665477 |
| F. Veillonellaceae.UCG | 0.03337796922 |
| Lactobacillus vaginalis | 0.02177078911 |
| Peptoclostridium spp. | 0.0373821786 |

### 7.1 Combinations of Taxa

Next, in order to assess possible combinations of taxa included in the list of taxa that it was found as differentially abundant according to the diagnosis (41) and to the clinically relevance (34) as potential candidates for the classification we used our validation set (100 extra samples from the colorectal cancer Catalan screening). It was identified a total of 27 taxa intersecting between the CRIPREV project and these extra samples, that are the ones included in the results presented here.

Different combinations of the taxa were assessed, considering the effect size observed in our statistical test (the one presented here, that detected them as dysregulated according to the variables of interest). It was defined top and down taxa from the list and it was made an assessment of subsets of taxa as follows:
4 taxa from the top of the list (50 random combinations)
4 taxa from the bottom of the list (50 random combinations)
4 random taxa (50 random combinations)
2 taxa from the top of the list (all the possible combinations)
2 taxa from the bottom of the list (all the possible combinations)
1 taxa from the top of the list (all the possible combinations)
1 taxa from the bottom of the list (all the possible combinations)

It was assessed possible subsets of taxa with classification potential (i.e., being differentially abundant in the invention differential analysis test) by using 100 extra samples from the same local screening. It was assessed different combinations of the taxa, considering the effect size observed in the invention statistical test. It was defined top (having high size effect) and down (having low size effect) taxa from the list, per each phase, and it was made an assessment of subsets of taxa as follows: 4 taxa from the top of the list (50 random combinations), 4 taxa from the bottom of the list (50 random combinations), 4 random taxa (50 random combinations), 2 taxa from the top of the list (all the possible combinations), 2 taxa from the bottom of the list (all the possible combinations), 1 taxa from the top of the list (all the possible combinations) and 1 taxa from the bottom of the list (all the possible combinations). From FIG. 8 it can be seen that both Akkermansia spp. and Akkermansia muciniphila are the ones with highest effect size in the group of differentially abundant taxa that are overrepresented in CRC.

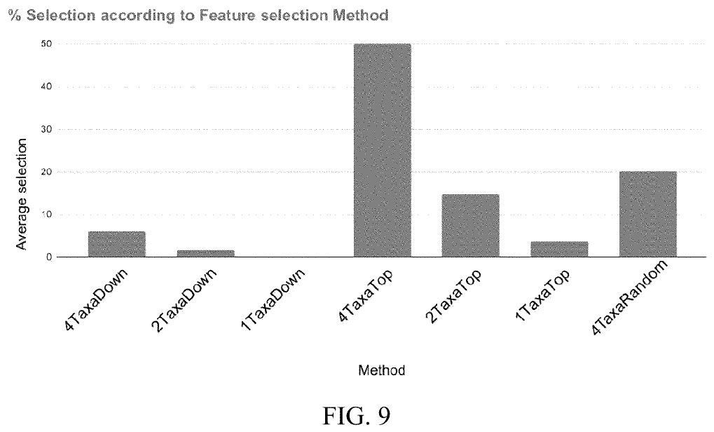

It was tested a total of 948 models using the validation set. It was filtered the models based on some metrics (AUC1>=0.55, Specificity>0.2, AUC2>0.5 and Specicity2>0) selecting 13.5% of the models (128/948). The strategy that selected more models is the one including subsets of 4 taxa with highest effect size (FIG. 9).

The selected models were divided in three grades, considering their predictivity values:

Grade 1 included 8 models with 100% sensitivity for CRC, >=96% sensitivity for Clinically relevant individuals and >=12% discarded unnecessary colonoscopies. The list of grade 1 combinations are:

Phase1 {Akkermansia.muciniphila, Bacteroides.plebeius}, Phase2 {Bacteroides.coprocola,
Bacteroides.caccae}
Phase1 {Akkermansia.muciniphila, Bacteroides.fragilis}, Phase2 {Bifidobacterium.longum,
Alistipes.finegoldii}
Phase1 {Akkermansia.unclassified.S361, Bacteroides.fragilis}, Phase2 {Bifidobacterium.longum,
Alistipes.finegoldii}
Phase1 {Akkermansia.unclassified.S361, Sutterella.wadsworthensis}, Phase2
{Bacteroides.coprocola, Dorea.formicigenerans}
Phase1 {Akkermansia.unclassified.S361, Sutterella.wadsworthensis}, Phase2
{Bilophila.unclassified.S322, Dorea.formicigenerans}
Phase1 {Akkermansia.unclassified.S361, unclassified.unclassified.S358,
Ruminococcaceae_UCG.002.unclassified.S91, Bacteroides.plebeius}, Phase2
{Negativibacillus.unclassified.S269, Bacteroides.coprocola, Bifidobacterium.longum,
unclassified.unclassified.S306}
Phase1 {Akkermansia.unclassified.S361, Ruminococcaceae_UCG.002.unclassified.S91,
Bacteroides.plebeius, Bacteroides.fragilis}, Phase2 {Negativibacillus.unclassified.S269,
Bacteroides.coprocola, Bifidobacterium.longum, Bilophila.unclassified.S322}
Phase1 {Akkermansia.unclassified.S361, Akkermansia.muciniphila,
unclassified.unclassified.S358, Bacteroides.plebeius}, Phase2
{Negativibacillus.unclassified.S269, Bacteroides.caccae, Dorea.formicigenerans,
unclassified.unclassified.S306}

Grade 2 and 3 included 50 and 70 selected combinations respectively.

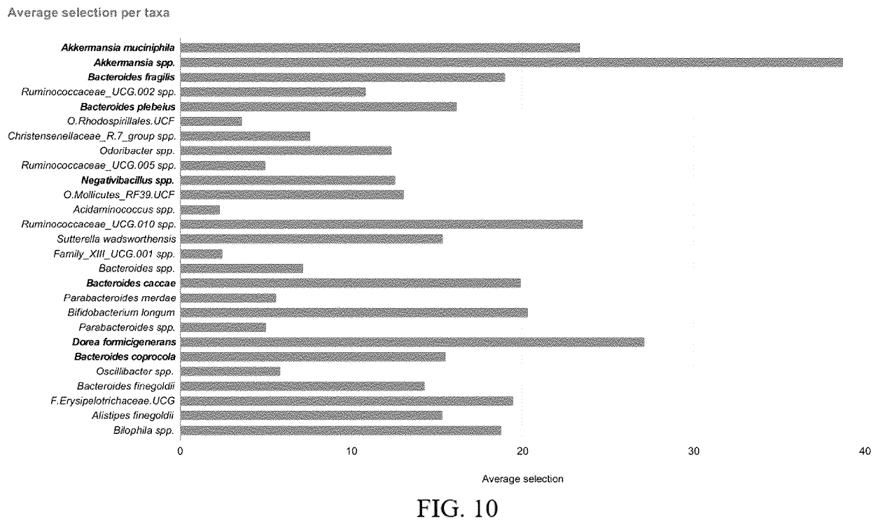

It was also explored the potential of the different 27 taxa by evaluating in how many models appeared each of them (FIG. 10, TABLE 5) being the taxa that appeared in most of the selected models Akkermansia spp.

TABLE 5
FOR EACH OF THE STUDIED TAXA: NUMBER
OF MODELS IN WHICH THE TAXA WAS INCLUDED,
AND NUMBER OF MODELS SELECTED.

| | Number | | |
|---|---|---|---|
| | models | Number | |
| | used as | selected | Average |
| Taxa | feature | models | selection |
| Akkermansia muciniphila | 214 | 50 | 23.36448598 |
| Akkermansia spp. | 217 | 84 | 38.70967742 |
| Bacteroides fragilis | 216 | 41 | 18.98148148 |
| Ruminococcaceae_UCG.002 spp. | 221 | 24 | 10.85972851 |
| Bacteroides plebeius | 229 | 37 | 16.15720524 |
| O. Rhodospirillales.UCF | 83 | 3 | 3.614457831 |
| Christensenellaceae_R.7_group spp. | 79 | 6 | 7.594936709 |
| Odoribacter spp. | 81 | 10 | 12.34567901 |
| Ruminococcaceae_UCG.005 spp. | 80 | 4 | 5 |
| Negativibacillus spp. | 271 | 34 | 12.54612546 |
| O. Mollicutes_RF39.UCF | 214 | 28 | 13.08411215 |
| Acidaminococcus spp. | 86 | 2 | 2.325581395 |
| Ruminococcaceae_UCG.010 spp. | 17 | 4 | 23.52941176 |
| Sutterella wadsworthensis | 215 | 33 | 15.34883721 |
| Family_XIII_UCG.001 spp. | 81 | 2 | 2.469135802 |
| Bacteroides spp. | 139 | 10 | 7.194244604 |
| Bacteroides caccae | 196 | 39 | 19.89795918 |
| Parabacteroides merdae | 142 | 8 | 5.633802817 |
| Bifidobacterium longum | 192 | 39 | 20.3125 |
| Parabacteroides spp. | 139 | 7 | 5.035971223 |
| Dorea formicigenerans | 188 | 51 | 27.12765957 |
| Bacteroides coprocola | 200 | 31 | 15.5 |
| Oscillibacter spp. | 137 | 8 | 5.839416058 |
| Bacteroides finegoldii | 14 | 2 | 14.28571429 |
| F. Erysipelotrichaceae.UCG | 190 | 37 | 19.47368421 |
| Alistipes finegoldii | 189 | 29 | 15.34391534 |
| Bilophila spp. | 192 | 36 | 18.75 |

The taxa with less models selected are the ones with smaller effect size.

124 out of the 128 selected models included at least one of the 8 taxa (4 taxa per phase) included in the selected model of the present application: Akkermansia muciniphila, Akkermansia spp., Bacteroides fragilis and Bacteroides plebeius, Bacteroides coprocola, Negativibacillus spp., Dorea formicigenerans or Bacteroides caccae.

### 7.2 Development of a Two Phase Machine Learning Classifier.

### Evaluation and Validation of the Strategy in Fit Positive Samples

Given that samples with different diagnoses presented significant differences in terms of the abundances of different bacterial taxa, it was explored machine learning approaches to develop a sample classifier able to distinguish samples that would more likely benefit from a colonoscopy intervention (i.e., those having clinically relevant diagnoses).

For this, it was put the focus on achieving high sensitivity as opposed to high accuracy, as false negatives (i.e., persons with clinically relevant lesions that do not proceed to colonoscopy) are of higher medical concern as compared to false positives (persons with no lesions that undergo colonoscopy).

To derive this predictor, it was explored the effect of using different machine learning algorithms, and the use of feature selection to restrict the parameter set to all bacterial taxa that had been observed to show significant differences, or to only a few of them (see section “Materials and Methods”). When including more taxa, it was observed a better AUC and specificity (TABLE 6). This can be translated to better reduction of false-positive rates. On the other hand, when restricting to only a panel of taxa it was obtained better recall and sensitivity for CRC and CR samples but poor AUC and specificity (TABLE 7). However, in the context of the current screening there is still a satisfactory reduction of the false-positive rate with a good prioritization of relevant cases. It was achieved optimal results with a two-phase classifier trained to classify CRC samples in a first phase and any CR samples in a second phase. This final classifier considers information on Sex, Age and FIT value that would be accessible from the FIT test results, and abundances from two different subsets of four taxa (First phase: Akkermansia spp., Akkermansia muciniphila, Bacteroides fragilis and Bacteroides plebeius and Second phase: Negativibacillus spp., Bacteroides coprocola, Bacteroides caccae and Dorea formicigenerans). This classifier obtained 98.98% sensitivity for CRC samples and 97.98% for clinically relevant samples.

TABLE 6
PERFORMANCE OF THE TWO-PHASE MACHINE LEARNING PREDICTOR.
THE REPORTED VALUES ARE MEAN VALUES OBTAINED FROM THE 100
RANDOM SPLITS. INCLUDING 41 AND 34 TAXA FOR BOTH PHASE 1
AND PHASE 2, RESPECTIVELY, PLUS SEX, AGE AND FIT VALUE.
A)

| | | | | TWO-PHASE | |
|---|---|---|---|---|---|
| | | | | CLASSIFIER | |
| | | | | | Sensitivity |
| | | | | Sensitivity | for CR |
| | AUC | Recall | Specificity | for CRC | lesions |
| FIRST PHASE | 0.618836 | 0.8048148 | 0.4328572 | 97.59% | 95.91% |
| SECOND PHASE | 0.5488568 | 0.7279377 | 0.369776 | | |

B)

| | Average likelihood to be misclassified | |
|---|---|---|
| | (not classified as relevant) | Average sensitivity |
| IRL | 4.43% | 95.57% |
| HRL | 4.16% | 95.84% |
| CIS | 2.58% | 97.42% |
| CRC | 2.41% | 97.59% |

A) AVERAGE OF AREA UNDER THE CURVE (AUC), RECALL AND SPECIFICITY FOR EACH OF THE PHASES AND AVERAGE SENSITIVITY FOR CRC AND CR SAMPLES AT THE END OF THE TWO-PHASE CLASSIFICATION WERE REPORTED.
B) AVERAGE LIKELIHOOD TO BE MISCLASSIFIED AND AVERAGE SENSITIVITY FOR EACH OF THE DIFFERENT LESIONS WITHIN THE GROUP OF CLINICALLY RELEVANT SAMPLES.

TABLE 7
PERFORMANCE OF THE TWO-PHASE MACHINE LEARNING PREDICTOR.
THE REPORTED VALUES ARE MEAN VALUES OBTAINED FROM
THE 100 RANDOM SPLITS. INCLUDING A PANEL OF 4 TAXA
FOR EACH OF THE PHASES PLUS SEX, AGE AND FIT VALUE.
A)

| | | | | TWO-PHASE | |
|---|---|---|---|---|---|
| | | | | CLASSIFIER | |
| | | | | | Sensitivity |
| | | | | Sensitivity | for CR |
| | AUC | Recall | Specificity | for CRC* | lesions |
| FIRST PHASE | 0.565368 | 0.8709974 | 0.2597385 | 98.98% | 97.98% |
| SECOND PHASE | 0.5358411 | 0.8052662 | 0.2664159 | | |

B)

| | Average likelihood to be misclassified | |
|---|---|---|
| | (not classified as relevant) | Average sensitivity |
| IRL | 2.29 | 97.71 |
| HRL | 1.94 | 98.06 |
| CIS | 1.46 | 98.54 |
| CRC | 1.02 | 98.98 |

A) AVERAGE OF AREA UNDER THE CURVE (AUC), RECALL AND SPECIFICITY FOR EACH OF THE PHASES AND AVERAGE SENSITIVITY FOR CRC AND CR SAMPLES AT THE END OF THE TWO-PHASE CLASSIFICATION WERE REPORTED.
B) AVERAGE LIKELIHOOD TO BE MISCLASSIFIED AND AVERAGE SENSITIVITY FOR EACH OF THE DIFFERENT LESIONS WITHIN THE GROUP OF CLINICALLY RELEVANT SAMPLES.

This strategy was validated by constructing a model with all the samples (without including Bacteroides fragilis) and testing it on an independent cohort of 135 FIT-positive samples from USA. The results of this adjusted model in the USA cohort yielded 100% sensitivity for CRC and 98.46% for CR lesions, reducing a 20% of the unnecessary colonoscopies (A). It was also made a validation with an independent dataset composed of 100 extra samples from the same Catalan Screening detecting all CRC samples, 96% of the CR samples and having a reduction of 12% of the false positives (TABLE 8).

TABLE 8
PERFORMANCE OF THE TWO-PHASE MACHINE LEARNING PREDICTOR
ON INDEPENDENT DATASETS. THE REPORTED VALUES ARE OBTAINED
BY TRAINING ON ALL THE CRIPREV SAMPLES (SAMPLES WITH MISSING
METADATA WERE DISCARDED FOR TRAINING THE MODEL, N =
2,817) AND TESTING ON THE INDEPENDENT SETS. AREA UNDER
THE CURVE (AUC), RECALL AND SPECIFICITY FOR EACH OF THE
PHASES AND SENSITIVITY FOR CRC AND CR LESIONS AT THE END
OF THE TWO-PHASE CLASSIFICATION WERE REPORTED.

| | | | TWO-PHASE CLASSIFIER | | |
|---|---|---|---|---|---|
| | | | Sensitivity | Sensitivity | Saved |
| | | | for CRC | for CR lesions | colonoscopies |
| AUC | Recall | Specificity | (%) | (%) | (%) |

A)

| | | | | | | |
|---|---|---|---|---|---|---|
| FIRST PHASE | 0.5721 | 0.9518 | 0.1923 | 100 | 98.46 | 20 |
| SECOND PHASE | 0.9231 | 0.8462 | 1 | | | |

B)

| | | | | | | |
|---|---|---|---|---|---|---|
| FIRST PHASE | 0.5789 | 0.8125 | 0.3452 | 100 | 96 | 12 |
| SECOND PHASE | 0.5952 | 0.8571 | 0.3333 | | | |

A) USA COHORT. INCLUDING A PANEL OF 3 AND 4 TAXA FOR PHASE 1 AND 2, RESPECTIVELY, PLUS SEX, AGE AND FECAL HEMOGLOBIN CONCENTRATION.
B) 100 EXTRA SAMPLES FROM THE CATALAN SCREENING.

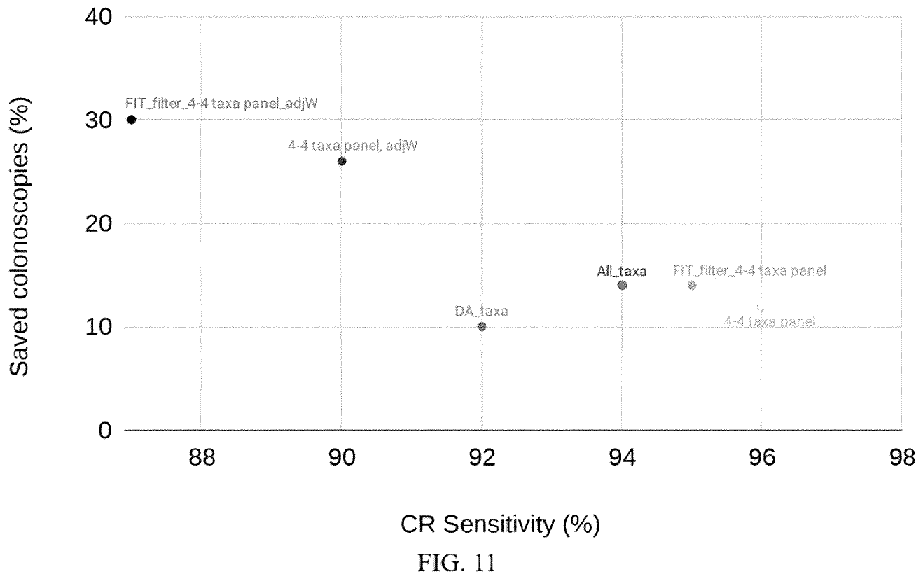

Taking profit of the balanced 100 extra samples from the Catalan screening, it was explored how changing some parameters of the classifier affected sensitivity and the number of saved colonoscopies. For instance, by penalizing less the minority class (CR) at the second phase, it was obtained better reduction of unnecessary colonoscopies (26%) but at the cost of including less CR samples (90%).Similarly, the number of samples to be tested can be reduced by applying a FIT-value threshold above which a benefit of colonoscopy is assumed. Applying a value of 954 μg hemoglobin/g feces (3rd quartile in CR samples) for such a threshold, which is passed by 18% of our samples, would save 14% of unnecessary colonoscopies at the end of the process. When we combined both approaches, it could reach 30% of saved colonoscopies, at the cost of a reduction of CR detection (87%). However, in all the mentioned cases it was detected 100% of the CRC samples. This shows that the algorithm can be fine-tuned to optimize cost-effectiveness (FIG. 11).

While certain representative embodiments and details have been shown to illustrate the present invention, it will be apparent to those skilled in this art that various changes and modifications can be made and that, within the scope of the appended claims, the invention may be practiced otherwise than as specifically described and claimed.

Example 8. Microbiome in Fit Negative Samples. Further Validation of the Two-Phase Classifier.

The differential analysis resulted in 39 taxa having a significant result (P value<0.05) when comparing CRC vs the others and 42 taxa as differential abundant according to CR vs Non-CR for the second phase.

It was also evaluated the machine learning classifier strategy by using this dataset, following the same scheme of the FIT positive samples shown above but considering the taxa found as differential abundant in this case. For the first phase it was included “Fusobacterium.unclassified.S106”, “Peptostreptococcus.unclassified.S87”, “Erysipelotrichaceae_UCG.003.unclassified.S297”, “Alistipes.putredinis”, sex and age. For the second phase, it was included “Prevotella.unclassified.S33”, “Akkermansia.unclassified.S361”, “Coprococcus.comes”, “Bifidoba cterium.longum”, sex and age.

It was applied the optimal strategy for the two-phase classifier (including 4 taxa per phase and clinical variables). It was evaluated the strategy by training and testing 100 models (to avoid lucky splits when creating the training and test sets). The results that were obtained are shown at the following table:

TABLE 9
PERFORMANCE OF THE TWO-PHASE MACHINE LEARNING
PREDICTOR IN FIT NEGATIVE SAMPLES. THE REPORTED
VALUES ARE MEAN VALUES OBTAINED FROM THE 100
RANDOM SPLITS. INCLUDING A PANEL OF FOUR TAXA
FOR EACH OF THE PHASES PLUS SEX AND AGE.

| | AUC | Recall | Specificity |
|---|---|---|---|
| FIRST PHASE | 0.7003007 | 0.8031454 | 0.597456 |
| SECOND PHASE | 0.5603614 | 0.682484 | 0.4382388 |

AUC: AVERAGE OF AREA UNDER THE CURVE.

The sensitivity for CRC and CR at the end of the procedure was:
Sensitivity CRC at the end of the two-step procedure: 98.38
Sensitivity CR at the end of the two-step procedure: 95.73913

This shows that the method of the invention is predictive for FIT-negative samples.

### REFERENCES

1. Bray, F. et al. Global cancer statistics 2018: GLOBOCAN estimates of incidence and mortality worldwide for 36 cancers in 185 countries. CA Cancer J. Clin.68, 394-424 (2018).
2. Hong, S. N. Genetic and epigenetic alterations of colorectal cancer. Intest Res16, 327-337 (2018).
3. Valle, L. et al. Update on genetic predisposition to colorectal cancer and polyposis. Mol. Aspects Med.69, 10-26 (2019).
4. Murphy, N. et al. Lifestyle and dietary environmental factors in colorectal cancer susceptibility. Mol. Aspects Med.69, 2-9 (2019).
5. Saus, E., Iraola-Guzman, S., Willis, J. R., Brunet-Vega, A. & Gabaldón, T. Microbiome and colorectal cancer: Roles in carcinogenesis and clinical potential. Mol. Aspects Med.69, 93-106 (2019).
6. Zou, S., Fang, L. & Lee, M.-H. Dysbiosis of gut microbiota in promoting the development of colorectal cancer. Gastroenterol. Rep.6, 1-12 (2018).
7. Zackular, J. P., Rogers, M. A. M., Ruffin, M. T., 4th & Schloss, P. D. The human gut microbiome as a screening tool for colorectal cancer. Cancer Prev. Res.7, 1112-1121 (2014).
8. Sheng, Q.-S. et al. Comparison of Gut Microbiome in Human Colorectal Cancer in Paired Tumor and Adjacent Normal Tissues.Onco. Targets. Ther.13, 635-646 (2020).
9. Yu, J. et al. Metagenomic analysis of faecal microbiome as a tool towards targeted non-invasive biomarkers for colorectal cancer. Gut66, 70-78 (2017).
10. Winawer, S. J. The history of colorectal cancer screening: a personal perspective. Dig. Dis. Sci.60, 596-608 (2015).
11. Young, G. P., Rabeneck, L. & Winawer, S. J. The Global Paradigm Shift in Screening for Colorectal Cancer.Gastroenterology 156, 843-851.e2 (2019).
12. Zou, S., Fang, L. & Lee, M.-H. Dysbiosis of gut microbiota in promoting the development of colorectal cancer.Gastroenterol. Rep.6, 1-12 (2018).
13. Vega, P., Valentin, F. & Cubiella, J. Colorectal cancer diagnosis: Pitfalls and opportunities. World J. Gastrointest. Oncol.7, 422-433 (2015).
14. Inici.http://www.prevenciocolonbcn.org/ca/.
15. Alix-Panabières, C. & Pantel, K. Circulating tumor cells: liquid biopsy of cancer.Clin. Chem.59, 110-118 (2013).
16. Bettegowda, C. et al. Detection of circulating tumor DNA in early- and late-stage human malignancies.Sci. Transl. Med.6, 224ra24 (2014).
17. Duran-Sanchon, S. et al. Identification and Validation of MicroRNA Profiles in Fecal Samples for Detection of Colorectal Cancer.Gastroenterology 158, 947-957.e4 (2020).
18. Nannini, G., Meoni, G., Amedei, A. & Tenori, L. Metabolomics profile in gastrointestinal cancers: Update and future perspectives. World J. Gastroenterol.26, 2514-2532 (2020).
19. Thomas, M. et al. Genome-wide Modeling of Polygenic Risk Score in Colorectal Cancer Risk. Am. J. Hum. Genet. 107, 432-444 (2020).
20. Janney, A., Powrie, F. & Mann, E. H. Host-microbiota maladaptation in colorectal cancer. Nature585, 509-517 (2020).
21. Sepich-Poore, G. D. et al. The microbiome and human cancer. Science371, eabc4552 (2021).
22. Quintero, E. et al. Colonoscopy versus fecal immunochemical testing in colorectal-cancer screening. N. Engl. J. Med.366, 697-706 (2012).
23. Atkin, W. S. et al. European guidelines for quality assurance in colorectal cancer screening and diagnosis. First Edition—Colonoscopic surveillance following adenoma removal. Endoscopy44 Suppl 3, SE151-63 (2012).
24. Click, B., Pinsky, P. F., Hickey, T., Doroudi, M. & Schoen, R. E. Association of Colonoscopy Adenoma Findings With Long-term Colorectal Cancer Incidence. JAMA319, 2021-2031 (2018).
25. Willis, J. R. et al. Citizen science charts two major ‘stomatotypes’ in the oral microbiome of adolescents and reveals links with habits and drinking water composition. Microbiome6, 218 (2018).
26. Willis, J. R. et al. Oral microbiome in down syndrome and its implications on oral health. J. Oral Microbiol.13, 1865690 (2020).
27. Callahan, B. J. et al. DADA2: High resolution sample inference from amplicon data. doi: 10.1101/024034.
28. Quast, C. et al. The SILVA ribosomal RNA gene database project: improved data processing and web-based tools. Nucleic Acids Res.41, D590-6 (2013).
29. Schliep, K. P. phangorn: phylogenetic analysis in R. Bioinformaticsvol. 27 592-593 (2011).
30. Wright, E., Erik & Wright, S. Using DECIPHER v2.0 to Analyze Big Biological Sequence Data in R. The R Journalvol. 8 352 (2016).
31. McMurdie, P. J. & Holmes, S. phyloseq: an R package for reproducible interactive analysis and graphics of microbiome census data. PLOS One8, e61217 (2013).
32. Gloor, G. B. & Reid, G. Compositional analysis: a valid approach to analyze microbiome high-throughput sequencing data. Can. J. Microbiol.62, 692-703 (2016).
33. Palarea-Albaladejo, J. & Martin-Fernández, J. A. zCompositions—R package for multivariate imputation of left-censored data under a compositional approach. Chemometrics and Intelligent Laboratory Systemsvol. 143 85-96 (2015).
34. Gloor, G. B., Macklaim, J. M., Pawlowsky-Glahn, V. & Egozcue, J. J. Microbiome Datasets Are Compositional: And This Is Not Optional. Frontiers in Microbiologyvol. 8 (2017).
35. Mas-Lloret, J. et al. Gut microbiome diversity detected by high-coverage 16S and shotgun sequencing of paired stool and colon sample. Sci Data7, 92 (2020).
36. Babraham Bioinformatics—FastQC A Quality Control tool for High Throughput Sequence Data. https://www.bioinformatics.babraham.ac.uk/projects/fastqc/.
37. Bolger, A. M., Lohse, M. & Usadel, B. Trimmomatic: a flexible trimmer for Illumina sequence data. Bioinformaticsvol. 30 2114-2120 (2014).
38. Wood, D. E., Lu, J. & Langmead, B. Improved metagenomic analysis with Kraken 2.Genome Biol.20, 257 (2019).
39. Lu, J., Breitwieser, F. P., Thielen, P. & Salzberg, S. L. Bracken: estimating species abundance in metagenomics data. PeerJ Computer Sciencevol. 3 e104 (2017).
40. Hmisc: Harrell Miscellaneous. https://CRAN.R-project.org/package=Hmisc.
41. Bates, D., Mächler, M., Bolker, B. & Walker, S. Fitting Linear Mixed-Effects Models Using lme4. J.Stat.Softw.67, (2015).
42. Fox, J., Friendly, M. & Weisberg, S. Hypothesis Tests for Multivariate Linear Models Using the car Package. The R Journalvol. 5 39 (2013).
43. Hothorn, T., Bretz, F. & Westfall, P. Simultaneous inference in general parametric models. Biom. J.50, 346-363 (2008).
44. Rivera-Pinto, J. et al. Balances: a New Perspective for Microbiome Analysis. mSystems3, (2018).
45. Kurtz, Z. D. et al. Sparse and Compositionally Robust Inference of Microbial Ecological Networks. PLOS Computational Biologyvol. 11 e1004226 (2015).
46. Woloszynek, S. et al. Exploring thematic structure and predicted functionality of 16S rRNA amplicon data. PLOS ONEvol. 14 e0219235 (2019).
47. Kuhn, M. Building Predictive Models in R Using the caret Package. J.Stat.Softw.28, (2008).
48. Baxter, N. T., Koumpouras, C. C., Rogers, M. A. M., Ruffin, M. T., 4th & Schloss, P. D. DNA from fecal immunochemical test can replace stool for detection of colonic lesions using a microbiota-based model. Microbiome4, 59 (2016).
49. Abrahamson, M., Hooker, E., Ajami, N. J., Petrosino, J. F. & Orwoll, E. S. Successful collection of stool samples for microbiome analyses from a large community-based population of elderly men. Contemp Clin Trials Commun7, 158-162 (2017).
50. Feng, Y. et al. An examination of data from the American Gut Project reveals that the dominance of the genus Bifidobacterium is associated with the diversity and robustness of the gut microbiota. Microbiologyopen8, e939 (2019).
51. Yang, T.-W. et al. Enterotype-based Analysis of Gut Microbiota along the Conventional Adenoma-Carcinoma Colorectal Cancer Pathway. Sci. Rep.9, 1-13 (2019).
52.Sweeney, T. E. & Morton, J. M. The human gut microbiome: a review of the effect of obesity and surgically induced weight loss. JAMA Surg. 148, 563-569 (2013).
53. Rinninella, E. et al. What is the Healthy Gut Microbiota Composition? A Changing Ecosystem across Age, Environment, Diet, and Diseases. Microorganisms7, (2019).
54. Shussman, N. & Wexner, S. D. Colorectal polyps and polyposis syndromes. Gastroenterol. Rep.2, 1-15 (2014).

1. A method for diagnosing a subject to suffer from colorectal cancer (CRC) or classifying a subject to have higher risk for developing CRC in a patient cohort comprising:
(i) determining in a fecal sample isolated from a subject the levels of three or more bacterial taxa;
(ii) classifying with a computer algorithm in a first phase CRC samples vs. non-CRC samples using two or more bacterial taxa that are differentially abundant in CRC samples relative to non-CRC samples, the hemoglobin content of the sample, and the age and sex of the donor;
(iii) classifying with a computer algorithm in a second phase the samples that are classified as being non-CRC in the first phase into clinically relevant (CR) samples and non-CR samples using two or more bacterial taxa that are differentially abundant in CR samples relative to non-CR samples, the hemoglobin content of the sample, and the age and sex of the donor, wherein CR comprises intermediate risk lesions, high risk lesions, carcinoma in situ (CIS), and Colorectal cancer (CRC);
wherein the three or more bacterial taxa in step (i) are selected from the group consisting of Hungatella spp. Colinsella spp., Tyzzerella spp., Phascolarctobacterium succinatutens, Lactobacillus spp., Akkermansia spp., Akkermansia muciniphila, O. Mollicutes_RF39.UCF, Ruminococcaceae_UCG.002 spp., Ruminococcaceae_UCG.0010 spp., Odoribacter spp., O. Rhodospirillales.UCF, Victivallis spp, Ruminococcaceae_UCG.005 spp., Negativibacillus spp., Christensenellaceae_R.7_group spp., Oxalobacter spp., Butyrivibrio spp., Family_XIII_UCG.001 spp., Gemella spp., Peptostreptococcus spp., Pediococcus spp., Lactobacillus vaginalis, Enorma massiliensis, Megamonas funiformis, Peptostreptococcus anaerobius, Peptoniphilus lacrimalis, Lactobacillus oris, Alloscardovia omnicolens, Allisonella histaminiformans, Acidaminococcus fermatans, Collinsella bouchesdurhonensis, Corynebacterium spp., Veillonella dispar, Ezakiella spp., O. Chloroplast.UCF, Sphingomonas spp., Dialister succinatiphilus, Finegoldia magna, Bacteroides coprophilus, Eggerthella spp., Acidaminococcus spp., Enterococcus spp., Sutterella wadsworthensis, Bacteroides fragilis, Bacteroides plebeius, Bacteroides coprocola, Bifidobacterium longum, Bilofila spp., Parabacteroides merdae, DTU08 spp., Oscillibacter spp., Parabacteroides goldsteinii, Parabacteroides spp., Bacteroides spp., Coprobacter secundus, Prevotella timonensis, Streptococcus parasanguinis, Peptostreptococcus anaerobius, Streptococcus sobrinus, Lachnospiraceae_FCS020_group bacterium, Bifidobacterium dentium, Porphyromonas spp., Lachnospiraceae_UCC.008 spp., Enterobacter spp., Hungatella hathewayi, Ezakiella spp., Leukonostoc spp., Parabacteroides johnsonii, Bacteroides finegoldii, Eisenbergiella spp., Alistipes finegoldii, F. Erysipelotrichaceae.UCG, Dorea formicigenerans, Bacteroides caccae, Fusobacterium.unclassified.S106, Peptostreptococcus.unclassified.S87, Erysipelotrichaceae_UCG.003.unclassified.S297, Alistipes.putredinis, Prevotella.unclassified.S33 and Coprococcus.comes.

2. The method according to any one of the preceding claims, wherein the fecal sample is a fecal immunochemical test (FIT) sample.

3. The method according to claim 2, wherein when the sample is FIT positive, the bacterial taxa are selected from the group consisting of Akkermansia spp., Akkermansia muciniphila, Bacteroides fragilis, Bacteroides plebeius, Negativibacillus spp., Bacteroides coprocola, Bacteroides caccae, and Dorea formicigenerans.

4. The method according to claim 3, wherein in the first phase of the method the levels of Akkermansia spp., Akkermansia muciniphila, Bacteroides fragilis and Bacteroides plebeius are determined to classify the subject to have CRC, and in the second phase the levels of Negativibacillus spp., Bacteroides coprocola, Bacteroides caccae and Dorea formicigenerans are determined to classify a subject to have a risk of developing CRC.

5. The method according to claim 4, wherein in the first phase higher levels of Akkermansia spp. and/or Akkermansia muciniphila and lower levels of Bacteroides fragilis and/or Bacteroides plebeius are associated with CRC, and in the second phase higher levels of Negativibacillus spp. and/or Bacteroides coprocola and/or lower levels of Bacteroides caccae and/or Dorea formicigenerans are associated with a risk of developing CRC.

6. The method according to claim 5, wherein in the first and second phase,
if a first ratio comprising the centered-log ratios (clr) of the following taxa
$$\frac{Akkermansia spp . + Akkermansia muciniphila}{Bacteroides fragilis + Bacteroides plebeius}$$
is higher than −0.5512273;
and a second ratio
$$\frac{Bacteroides coprocola + Negativibacillus spp .}{Dorea formicigenerans + Bacteroides caccae}$$
is higher than 0,
the subject is diagnosed to have a risk of developing CRC.

7. The method according to claim 6, wherein when the sample is FIT negative, the bacterial taxa are selected from the group consisting of Fusobacterium.unclassified.S106, Peptostreptococcus.unclassified.S87, Erysipelotrichaceae_UCG.003.unclassified.S297, Alistipes.putredinis, Prevotella.unclassified.S33, Akkermansia.unclassified.S361, Coprococcus.comes, Bifidobacterium.longum are determined to classify a subject to have a risk of developing CRC.

8. The method according to claim 7, wherein in the first phase higher levels of Fusobacterium.unclassified.S106, Peptostreptococcus.unclassified.S87, Erysipelotrichaceae_UCG.003.unclassified.S297 and Alistipes.putredinis, and in the second phase higher levels of Prevotella.unclassified.S33, Akkermansia.unclassified.S361, Coprococcus.comes and Bifidobacterium.longum, are determined to classify a subject to have a risk of developing CRC.

9. The method according to claim 1, wherein a subject classified in a cohort of subjects as having risk of developing CRC in step (iii) is considered to require a colonoscopy, and those subjects not classified in a cohort of subjects as having risk of developing CRC in step (iii) are considered to not require a colonoscopy.

10. The method according to claim 1, wherein the computer algorithm is selected from the group consisting of an artificial intelligence algorithm, a machine learning algorithm, and a trained neural network algorithm.

11. The method according to claim 10, wherein the computer algorithm is a trained neural network algorithm.

12. A kit comprising:
(a) reagents for conducting a method for determining the presence or the abundance of the bacteria in a fecal sample to determine the levels of two or more bacterial taxa in step (i) of the method of claim 1; and
(b) a computer program stored on a computer-readable data carrier or chip, comprising instructions which, when the program is executed by a computer, cause the computer to carry out steps (ii) and (iii) of the method of claim 1.

13. The kit according to claim 12, wherein the reagents are for conducting 16S rRNA gene sequencing.


SEQUENCE LISTING (2 sequences)

SEQ ID NO: 1 - DNA, 50 bp, synthetic construct
  source 1..50: PCR_primers = fwd_name: V3_V4_Forward, fwd_seq:tcgtcggcagc gtcagatgtgtataagagacagcctacgggnggcwgcag, rev_name: V3_V4_Reverse, rev_seq:gtctcgtgggctcggagatgtgtataagagacagg actachvgggtatctaatcc; mol_type = other DNA
  variation 46: note = w = a or t
  variation 42: note = n = a, c, g or t
tcgtcggcagcgtcagatgtgtataagagacagcctacgggnggcwgcag

SEQ ID NO: 2 - DNA, 55 bp, synthetic construct
  source 1..55: PCR_primers = fwd_name: V3_V4_Forward, fwd_seq:tcgtcggcagc gtcagatgtgtataagagacagcctacgggnggcwgcag, rev_name: V3_V4_Reverse, rev_seq:gtctcgtgggctcggagatgtgtataagagacagg actachvgggtatctaatcc; mol_type = other DNA
  variation 41: note = h = a, or c, or t
  variation 42: note = v = a or c or g
gtctcgtgggctcggagatgtgtataagagacaggactachvgggtatctaatcc